SEQUENCE LISTING (19 sequences)

SEQ ID NO: 1 - DNA, 16101 bp, Sorghum bicolor
  source 1..16101: mol_type = genomic DNA
gtgtggtgcagccgtcaaaatgtccagccgtccaaacacacgccaagagcgcgcgcagagcacccccacccagcaacccaaaaaccctcgggccctgccggaggaatggaaagggacacgccaggaaatcccctccccacccagcccaggccacgccccgtcatcacatcagaaaccacgcgcgtcccgtcccgtcccgtcccgtccgatctcccccccaccctatccacccggcggcatccgggccctccacttgtccccaccgttatctcccccctgtcacgtgccgctgccgctgctgcgctccaccgcggcggagtccccaatgccgatgcgagccgcgaaatttatttatcataggccgcacagcccccatctctcctagtcctgcctgcctgcctgcctcgccccagtcgccctcaccaccccacccccaacccccctcgggatatccgcctctgctccgctccgcccggcctcctcctcctcctcagctccctccgcctccgcctccgccgccacaaggatccaacccgcgcgcgtcgccttctccgccccccgccgcctttcatcccgaggtaccagctcgccccccccgtcctccctcctcctccgcttatttattccgccgtgtcatcgccagcgccgctcgctccttctgtcgcggccggccaagtggagctcgtcagatccgcgcgccgcacgccgatccgcgccaccgacggtgagaaccgcgcgcccgccaatggccccctcttcgttgctccgcctatgtatacagggtttaagctagggggtggggggtggggttcgtcagcgccagccgcgggggagggtttttgcatgcgccaccccaccccgaccggaagagcggaattccgtgagcctgtacggcaatggcaatggtggtggggttagttactagggtgggtgaatggtttcgtcgggttataataatactaataacacgaattgtttgtgcccacctgcgctgcgcgggcgaaatgtaggttgtctcttgttgcattgccagatggatgaggtagtctctggctgcctctgctagtctctagcgttgctgactggctgtggtctattcagggatgcccatgcccatgcccatgctagtagattgctgggtaccgttcgttcttgtctgtacatggttggtggcgggtctgcgtctttgttgtcactgaggtttaaggtttattaaggtgtagtagtatcagtggctaccctcatgacttaaggtttatttattttaacacacctgtttctttgtgtgggtacataaggaatgcgcttggttttgttccctgttagttttcgagcatgtgtcattctaccaagtgggttactgtaacattgcaccctttgatggttggtggttcttcatcttttctcctaccttggttatctaatacctgcgtgtaactgatgacctacttttttatatcatatagtttattacatctttttgtggtacatatcaaatctgaaaaataatgttttgtgttcttagtatgttgtgcacaaaataaaaggtggtttcacctgcaacctttgaagaaaaaaaaaactttctgaaaaggagtgatcatttagtatgaagaaaatttgatttactttcttcagagggaatatttcaaaccaacaattttcttactgtccaagccactgaaattttatcatgatcttacatgcacgagccacatgaaaccttatcaccccctgataaaaaaaaagaaaggatcaatttggtgattcatagaatggggaaaaacaacctgttgccatttggggagcttgttgtatactcaggttggggtccttgtccctgctcccttcctatgcaaccatctccattgaaaagattttgcactacatttgggctttgtatgataaaaaaaatctaaacagcagaaacatagtagaattgtagaaaaaaggtgctggcaagttttagtggcagagacctttgtatgtttggacatagcttactagtatatagttttctgtaatcttcatgaaaggtggccaataaacctgataagatctcaacatggtaggttccttcaaaatgagaggaaaactggaaacatcacaaatatttttctagcaactggcctataaaccataatgttgcttttcatttctttgatattcgaaacttcctaagagtattctgctagagttctgatggtattatttgcctctgtcagattttccaggaattttcctctctttttatggcactgtgcttttgagaagatctttaattgtgctgtctgggccacggaacgacaatgtcgcagcttggattagctgcagctgcctcaaaggcactgccactactccctaatcgccagcgaagtcctgctgggactacattctcatcatctgcattgtcgaggccctcaaaccgaaggaaaagccgtacccgttcactccgtgatggtggagatggggtatcagatgtcaaaaagcacagccagtctgttcgtcaaggtactgtgaatatctttcgatacaagctaaaattttgtttcaaaatatatatggagttctttattggctggcattgttcatttgttttaacatcgtaatgtttgatgcctcattctaggtatcaacgtagatatgctcactgctcagtttcaaatgtttgtctgcatgtaggtcttgctggcattattgacctcccaagcgaggcagcttcggaagtggacatttcacagtaagcggctatagtattttgcgtattatatgtttgttttggaaaaagaaaatattctgcagcttatttatactagtttccctgatagtgaaatgctgtcttaatgtcctagtgctgtatactcaatatttcgtagtaaatgctgcaaaatatgtgatataaatgttgcaacacagccaggggcctgcttctgtacctgctattgagcatgatgaatacttcgctcacttatatgatgtggttatatctcatgtagaagaattagttgcagtattgctggacaatagttacttattatgaaatcatgtctgctacaaattttctggcttctgttgctgtaaatgcctgtttttttttttttggaaaagattaaatgtttgacctaaattggacatatactccctccgtccctgaatatctgtcgttctcgcttcccgagaaatgacttttattaattttatataaaaacatattaatattttttatacaattgacatcattagataggtctttaatctagttttttaataaatttatttggagatataaatgttgtacgcatttgctacaaatctagtcaaacttgtggcaggaaacccagaaacgacacttaaaaaggggcggagggagtatgatagacgcaaagctcattttgaacttttttgatttgtgacaaccatgcatattgttagtgttgtgaacaaaatggtaatcctttccttttgtatttttccagtggatctgaggatcctaggggtccaccagattcctatcaaatgaatgggattatcaatgaaacacataatggaagacatgcttcagtgtccaaggttgttgaattttgtgcggcactaggtggcaaaacaccaattcacagtatattagtggccaataatggaatggcagcagcaaagttcatgaggagtgtccggacatgggctaatgatacttttggatctgagaaggcaattcagctcatagctatggcaactccggaagacatgaggataaatgcagagcacattagaattgctgatcaatttgtagaggtgcctggtggaacaaacaataataactacgccaacgttcaactcatagtggaggttagcactgctaatctgttagtttactactgatcggctgttccctttatttcttctataaccattgtcatatttaagtagagaagtttatatttctcctctgctgttgttgtggaagtctaattgtcaccatttattaactatgaaatattgcaggtagcagaaagagtaggtgtttctgctgtttggcctggttggggtcatgcttctgagaatcctgaactgccggatgcattgactgcaaaaggaatcgttttccttgggccacctgcatcatcaatgaatgcattgggagataaggtcggttcagctctcattgctcaagcagctggggtcccaactcttgcttggagtggatcacatgtgagtctctctctctctctctctctctctctctctctttgattactatctgccggtctcattgctctaactttcatattctaataatgacactaaataggttgaagtgccattagagtgctgcttagacgctatacctgaggagatgtatagaaaagcttgtgttactaccacagaggaagcagttgcaagttgtcaagtgattggttatcctgccatgattaaggcatcctggggaggtggtggtaaaggaataagaaaggtgtgttttatttatgtgactaaactatctgtgaagaactgtgcacattggggagtatggtgtagggagctacccccctctcctgtgatgtgattaaatcaatttccctggcaggttcataatgatgatgaggttagagcactgtttaagcaagtacaaggtgaagtccctggctccccaatatttatcatgaggcttgcagcccaggttagttttttttttctttctgaaatctatattccatccctttttgttcttttaaagttatccttgtattttctggaagcttcatctgatgcattattgacaagtgcactgatgatcatcatatttggagattaacatatttatgaaaggttaattgatgggaactgttgaaagagaacggttgagcggataccattctatttttagaatttagaaatcgcggtttttgcgtacaaaattgcatttccaggaactggactaagcttttcttagtattgagtggcatgttatacatggaccatttttgtcaacttacagggctgtgataatggctggagagaaataatacatcttgtttctcaacacttatgtggagaagatgttttacctttttctaaaatcactttttggattaaattgtataagttttcaatattctcactattattgaaccaaaacatgtttcattctttacacctttatttttcaagatggaagcctggaattgtgctctgttatctatagtcaattagtcatatatttatttgattttaaatctttttctctattgtagagtcggcatcttgaagttcagttgctttgtgatcaatatggcaatgtagcagcacttcacagtcgtgattgcagtgtgcaacggcgacaccagaaggtctgcccccacccactcatccataaacaccaaattatagaaccatgcattttgttatgcgatctatttcttaactgtagttccattcgcatttttctacaacagattattgaagaaggcccagttactgttgctcctcgtgagacagttaaagcacttgagcaggcagcaaggaggcttgctaaggctgtgggttatgttggtgctgctactgttgagtatctttacagcatggaaactggagaatactattttctggagcttaatccccgactacaggtttgctatacgtgaacatcattgactaattaacctgtcggggaatccttcacataaaattatctccatgcaggtcgagcatccagtcaccgaatggatagctgaagtaaatctgcctgcagctcaagttgctgttggaatgggcatacctctttggcaaattccaggtaattaccaatttaccaacttatttagttccttattagtttattctctaatctgtctacttatgtagaaatcagacgtttctatggaatggactatggaggagggtatgatatttggaggaaaacagcagctcttgctacaccatttaattttgatgaagtagattctcaatggccaaagggccattgtgtagcagttagaattactagtgaggaccctgatgatggtttcaaacctactggtgggaaagtgaaggtaagattttgagatgacagatgtattatgtatagttcaaacagattaagtttggttaagtgaccagatcttgattttttatcttttaggagataacttttaaagccaagcctaatgtttgggcctacttctcagtaaaggtaacttgttaactttgttacactgttacattattcttcattgtgcagataatttggatgggactaagtttttaaccattcatcgtctcatttagctaagcaaatgtttgcactgactcccctttttatctgctttcagtctggtggaggcattcatgaatttgctgattctcagtttggtatgtgtaaatcaagaatattcttctttgtaatttgtattggtcctcattttctaaatattgctctttccgttacaggacatgtttttgcatatggactctctagatcagcagcaataacaaacatggctcttgcattaaaagagattcaaattcgtggagaaattcattcaaatgttgattacactgttgacctcttaaacgtatgaaatattaaccaccttttgaatccctgttttcattatgctgattcatatcattatgtttgattttccattatggctaaacctgtggtgctatttccctattattccaggcttcagacttcagagaaaacaagattcatactggttggctggataccagaatagctatgcgtgttcaagctgagaggcccccatggtatatttcagtggttggaggtgctttatacgtaagataaagaaatcatgctaacatcttttgtcaaactactgtgaaaaacatacgatgtaaggtctaatttaaataattgtcacatgctacagaaaacagtaaccaccaatgcagccactgtttctgactatgttagttatctcaccaagggccagattccaccaaaggtatgttttgtgggattaactctggatatttttaaggtgaaaaatggttgacgaataatatttttatgcagcatatatcccttgtcaattctacagttagcttgaatatagaagggagcaaatacacagtaagtttgacattccacaagggaatttattttagttgtaacaataaagttagacatcattctgagtttcgtctgcattgtgttgtagattgaaactgtaaggactggacatggtagctacaggttgcgaatgaatgaatcgacagttgaagcgaatgtacaatctttatgtgatggtggactcttaatgcaggtaacttgttctttcttttgtgcattattattaattagttggataaatggttttgatttcatggcggttctgattgttgaattgcaatggctccagttggatggaaacagccatgtaatttatgcagaagaagaagctggtggtacacggcttcagattgatggaaagacatgcttgttgcaggtaaatactcccttcctctatattcttggtgtctgattgggcaacttctgccagatttatctgtatcatttattattgcattttggtccactaccttatctttaaaagatgggttctgttgtttgcttccgcaggagacatcacataagaaaattgttactaatccttgttttcttgcagaatgaccatgatccatcaaagttattagctgagacaccctgcaaacttcttcgtttcttggttgctgatggtgctcatgttgatgcggatgtaccatacgctgaagttgaggttatgaagatgtgcatgcctctcctgtcacctgcttctggtgtcgttcattgtatgatgtctgagggccaggcgttgcaggttatattcttacatgtccattccttgcattgtgctttcatcccataatatttcatgtaacatttgtcaaattacatttgttttaggctggtgatcttatagcaaggctggatcttgatgacccttctgctgtgaaaagggctgaaccatttgatggaatatttccacaaatggcgctccctgttgctgcctctagtcaagtacacaaaagatatgctgcaagtttgaatgctgctcgaatggttcttgcaggatatgagcacaatatcaatgaagtaaacattccatcttattgtgaccacatcctgtttattttctttgtttatcttgtgtttcctttacttagatagtttctacaaaaatgatcttttgtccagtcacatgtcttctccctcactcatctacctatcatatctcttcatgtcttggtttccgtgtatacatggtttcttattaagaaaccatttccccctcttctttaattgtagtgccacatcatcatttctgtttagtttgcaccctaattaatgaacatataaaccatgtagtttctgggttgggagtgcccttagcacttggttatccattatcaactgattcatgttaagctattcttcttttcaggtcgttcaagatttgatatgctgcctggacaaccctgagcttcctttcctacagtgggatgaacttatgtctgttctagcaacgaggcttccaagaaatctcaagagtgaggtatgagacagttggcaagatatagccaatctggattaaaggaaagatgattcattttagtagatatattatcttaattttgcattttacatattggcttatgttgatggcagaaaaagccatcagagaaactgtgatattttataacattagtttgcaccgtgttatgctcataaataaacctgacaatggtttgggccggaaccagtttccatggtttgatttccttgttgggccaattcaatttgggtgaaaacgggctgagcaagctggtgggctggttcagtctgagttattacttagagtagtttagtttggtttggttttggactgagtgttttggcttgataacaggctgaagctgtggttccaacccagcatcacaccacggtacagcagaagccccatccgcgtccactagtattcctattgtattactggatttgtttttctataggaaaacggccataaagtgtagcatgacgtcatgctgatgttgacaagctaggaatgaagcagagcacacacaacactcagccggctactcgctccgcgccgcgcagcagcgcatgtcacgcatgcacatgtgggaggacgtgcacagacgtccgtgctcggctcggccacgctcgggacctcgtggttgccggagctgccgggagcggtgcgaatggcggcgcatggccggacggaggagaggctcgcgctgcactgcttgggctttgccagcctcaagacctccggggccgtcaagctcgtggatgccgcggtgctggcgcgctccctgcccaggctctactctctcttacctgctctccatcctctccagcttcagtgacctgcgggagttcatcatttcagcgcccagagctgtatcagcttctataacaaggacgaggaggtgctcaggcaaggggccaggatccagcggttcgacgtcagagggtccaagttggtggacgagatagagaatgagttcgccggtggtgaagttttctgcgactcgtcctacgttgaagtgatgtgatctctgtctgaaccattccacgatgacagttcgaagtcggtttcgaatcccggttgtaggctgtggactggtcttgtttggatttaggaacactacagtttggcgtggactggttttggagggggaacgagtggggtggattggggatttgaaggcagacgtgattagtttggtttggttatggtctggatcacggaccattaacaggtttactcataaaaatcgtttagttttttctgcagtttgttttgtaaaatttgatgaaaacttagaatttttcactctagcattttaccggaaggtgaatctttcagtaagtttgttttgattctgtgattgtagttctggtatgtccttacataatccatttttattctagcttctgatgtttttatcttttatgcatgcagttagaggataaatacaaggaatacaagttgaatttttaccgtggaaaaaacgtggacttcccttccaagttgctaagagacatcattgaggtcagttatgtttcttttgtggtatctcttaaactgtttgctattgtcttaatcttaactagtgttttctctgtaggaaaatcttgcatatggttcagagaaggaaaaggctacaaatgagaggcttgttgagcctcttacgaacctactgaagtcatatgagggtgggagagaaagtcatgcacattttattgccaagtcccttttcgaggagtatcttatggtggaagaacttttcagtgatggaattcaggttattgtacatgacaagactgaacagatcagtattagaaaaagcacacaaaatgttcactccttttcactctcttagttgggagaaactttttgcatggctagctgtatttattcgaacaatctgacttgttatttgtctctaattatgtcatggcagtctgacgttattgaaaccttgcgtcatcagcacagtaaagacctgcagaaggttgtagacattgtgttgtctcaccaggtaaatttctttatggccgatgacttttatgcaaatgattagtgaaacaatctttgttattgatagtgtgattttattcacagggtgtgagaaacaaagctaagcttgtaacagcactcatggaaaagctggtttatccaaatccggttgcttacagggatctgttggttcgcttttcttccctcaatcataaaagatattataaggtgcagtgattacaataacctagacatcccccttataatttattttgatgttttacctaatgaattccatgcatcttaataaacacagttggcccttaaagcaagtgaacttcttgaacaaaccaaactaagtgaactccgtgcaagcattgcaagaagcctttcagatctggggatgcataagggagacatgactattaaggatagcatggaagatttagtctctgccccattgcctgttgaagatgctcttatttctttgtttgattacagcgatgcaactgttcagcagaaagtgattgagacatacatatcacgattgtaccaggtattatatcaactaagttgatgtctttcatagtcccactaagcatatctgatatgttagatgccctaatgacatggaatgctcatctcttcatttcacatgaagaaatattgagaaatgagatgctgatgtttggctgaattaactgggtgtgaaaaattgtgatctcccaacttgttaatgcacaatgtgctggctaacttgccaatatatatttttttttcagcctcatcttgtgaaggatagcatccaaatgaaattcaaggaatatggtgctattgctttttgggaattttctgaagggcatgttgatacttcaaatggacatgggactattcttggtgggaagagatggggttccatggtcgtcctcaaatcacttgaatctgcatcaacagccattgtggctgcattaaaggattcagcacagtacaacagctctgagggcaacacgatgcacattgcattattgagtgctgaaaatgaaagtaatatgagtggaacaaggtttgttcatgtgcaatattaggtcgaaaggggtttttgtttaagttgtgtaactcttttttcttttaattgaggccatttctttcagcagtgatgatcaagctcaacataggatggaaaagcttaccaagatactgaaggatactaatgttgcaagtgatctccgagctgctggtttgaaggttataagttgcattgttcaaagagatgaagctcgcatgccaatgcgccacacattcctctggttggatgaaaagagttgttatgaagaagagcaaattctccggcatgtggagcctcccctctctgcacttcttgaattggtctgtttatcaaaatgattgcatgcttgtttgctacattctgttattatgctttgcttattttcttcactatacttattttgcctattatgaatacaggataagttgaaagtgaaaggatacaatgaaatgaagtatactccgtcacgtgatcgtcaatggcatatctacacactaagaaatactgaaaaccccaaaatgttgcatagggtatttttccgaactattgtcaggcaacccaatgcaggcaacaagtttacatcagctcaggtcagtgatactgaagtaggaggtcctgaagattctctgtcattcacatcgaatagcatcctaagatcattgatgactgctatagaagaattagagcttcatgcgattaggacaggtcattctcacatgtatttgtgcatactgaaagagcaaaagcttcttgatctcattccattttcagggtaagtgtgcacataatcctattcaaggaaatgtttatgcattgactattatatcggtctccttaacaagcttttcatattattcaggagtacaattgttgatgttggccaagatgaagctactgcttgttcacttttaaaatcaatggctttgaagatacatgaccttgttggtgcaaggatgcatcatctgtctgtatgccagtgggaggtgaaactcaagttggattgtgatgggcctgcaagtggtacctggagagttgtgactacaaatgttactagtcacacctgcaccattgatgtaagttgtccctactgttttagtattctgtatatcacacacatgaaagtataagtcaatgtgtggttcttatattctgaactgccatccatgtcattatgttctgaagcacatagtacttctaatggttttagatctaccgagaagtggaagatacagaatcgcagaagttagtataccattcaaccacttcggcagctggtcctggtccgttgcatggtgttgcactgaataatccatatcaacctttaagtgtgattgatctaaagcgctgctctgctaggaacaacagaacaacatattgctacgattttccgctggtgagttgacccccgccccacccctcagtgaatacacatctgtagttttttgaacctaagctgataatgaaaaaaaaatgtaggcatttgaaactgcactgcagaagtcatggcagtccaacggctctagtgtttctgtaggcagtggaaatagtaaatcctacgtgaaggcaactgagctggtgtttgctgaaaaacatgggtcctggggcactcctatagttcccatggaacgtcctgctgggctcaatgacattggtatggtcgcttggatcttggagatgtcaacacctgaatttcccaatggcaggcagattattgttgtagcaaatgatattactttcagagctggatcattcggcccaagggaagatgcattttttgaagctgtcaccaacctggcttgtgaaaggaaacttccccttatatacttggcagcaaactctggtgctaggattggcatagctgatgaagtaaaatcttgcttccgtgttgggtggtctgacgaaggcagccctgagcgagggtttcagtacatctatctgactgaagaagactatgcccgtattagctcttctgttatagcacataagctgcagctagatagcggtgaaattaggtggattattgactctgttgtgggcaaggaggatgggcttggtgttgagaacatacatggaagtgctgctatcgccagtgcttattctagggcatatgaggagacatttacacttacatttgtgaccggacggactgtaggaataggagcttatcttgctagacttggtatacggtgcatacagcgtcttgaccagccgattattttaacagggttttctgccctgaacaagctccttgggcgggaagtgtacagctcccacatgcagcttggtggtcctaagatcatggcgaccaatggtgttgtccacctgactgttccagatgaccttgaaggtgtttccaatatattgaggtggctcagctatgttcctgcaaacattggtggacctcttcctattaccaaacctttggaccctccagacagacctgttgcatacatccctgagaacacatgcgatccacgtgcagccatccgtggtgtagatgacagccaagggaaatggttgggtggtatgtttgacaaagacagctttgtggagacatttgaaggatgggcaaaaacagtggttactggcagagcaaagcttggaggaattcctgtgggtgtcatagctgtggagacacagaccatgatgcagcttgtccctgctgatccaggtcagcttgattcccatgagcgatccgttcctcgggctggacaagtgtggttcccagattctgcaaccaagacagctcaggcattattagacttcaaccgtgaaggattgcctctgtttatcctggctaactggagaggtttctctggtggacagagagatctctttgaaggaattcttcaggctgggtcaacaattgtcgagaaccttaggacatataatcagcctgcgtttgtctacattcctatggctggagagcttcgtggaggagcttgggttgtggtcgatagcaaaataaatccagaccgcattgagtgttatgctgagaggactgccaaaggtaatgttctcgaacctcaagggttaattgaaatcaagttcaggtcagaggaactccaagactgtatgggtaggcttgaccccgagttgataaatctgaaagcaaaactccaagatgtaaagcatggaaatggaagtctaccagacatagaatcccttcagaagagtatagaagcacgtacgaaacagttgctgcctttatatacccagattgcaatacggtttgctgaattgcatgatacttccctaagaatggcagctaaaggcgtgattaagaaagttgtagactgggaagaatcacgctctttcttctataaaaggctacggagaaggatctctgaagatgttcttgcaaaagaaataagacatatagtcggtgacaacttcactcaccaatcagcaatggagctcatcaaggaatggtacctggcttctccagccacagcaggaagcactggatgggatgacgatgatgcatttgttgcctggaaggacagtcctgaaaactacaatggatatatccaagagctaagggctcaaaaagtgtctcagtcgctctctgatctcactgactccagttcagatctacaagcattctcgcagggtctttctacgctattagataaggtaaatttgcttacagtttcaccttattttttatttgcaattaacctgtgggctacaaccggttgtatctgttgcatgttactttttaagagctcctatttcaattgcattgcatacatggcatcttatcaattcatgattctaatacatacccattgccttctcacgtattccgtctttggatgccgattcagatggatccctctcaaagagcgaagtttgttcaggaagtcaagaaggtccttggttgatgatatgataccaacacatccaacactatgtgcatgctacatgtttttgttcaagtacatacatagaaggatattgcttggcctcgtttgcttggccgtttgatcatgtctgatctaagtcgaccattatttgttgaaacttcctttttggacctggtgctatggttgatgaatgtatattggatgtgtgcgactgtgcgttctgccaggtgtaagctcaaatgtttagacagaccgagttatggttaggaagagcacgagtgaacatgttctggttttgcagtggttcaggaaggcagaaagttgtttcactgtagttctgagatgtactaccagcggccccctgctgtaattttagggtgtataatgcggatactagtaaaacaattgagtggttcattaaattttgaactcgaataatgtttttctaggcatatgtaccttacctctacgtgaaataaatgctgttgaaatagcattcgacaccagaatatatgtaccttacctaatatacagcttaacgtatggaccccatatgtcagtacttcatttttttttgtttttccactatcct

SEQ ID NO: 2 - protein, 2327 aa, Sorghum bicolor
MSQLGLAAAASKALPLLPNRQRSPAGTTFSSSALSRPSNRRKSRTRSLRDGGDGVSDVKKHSQSVRQGLAGIIDLPSEAASEVDISHGSEDPRGPPDSYQMNGIINETHNGRHASVSKVVEFCAALGGKTPIHSILVANNGMAAAKFMRSVRTWANDTFGSEKAIQLIAMATPEDMRINAEHIRIADQFVEVPGGTNNNNYANVQLIVEVAERVGVSAVWPGWGHASENPELPDALTAKGIVFLGPPASSMNALGDKVGSALIAQAAGVPTLAWSGSHVEVPLECCLDAIPEEMYRKACVTTTEEAVASCQVIGYPAMIKASWGGGGKGIRKVHNDDEVRALFKQVQGEVPGSPIFIMRLAAQSRHLEVQLLCDQYGNVAALHSRDCSVQRRHQKIIEEGPVTVAPRETVKALEQAARRLAKAVGYVGAATVEYLYSMETGEYYFLELNPRLQVEHPVTEWIAEVNLPAAQVAVGMGIPLWQIPEIRRFYGMDYGGGYDIWRKTAALATPFNFDEVDSQWPKGHCVAVRITSEDPDDGFKPTGGKVKEITFKAKPNVWAYFSVKSGGGIHEFADSQFGHVFAYGLSRSAAITNMALALKEIQIRGEIHSNVDYTVDLLNASDFRENKIHTGWLDTRIAMRVQAERPPWYISVVGGALYKTVTTNAATVSDYVSYLTKGQIPPKHISLVNSTVSLNIEGSKYTIETVRTGHGSYRLRMNESTVEANVQSLCDGGLLMQLDGNSHVIYAEEEAGGTRLQIDGKTCLLQNDHDPSKLLAETPCKLLRFLVADGAHVDADVPYAEVEVMKMCMPLLSPASGVVHCMMSEGQALQAGDLIARLDLDDPSAVKRAEPFDGIFPQMALPVAASSQVHKRYAASLNAARMVLAGYEHNINEVVQDLICCLDNPELPFLQWDELMSVLATRLPRNLKSELEDKYKEYKLNFYRGKNVDFPSKLLRDIIEENLAYGSEKEKATNERLVEPLTNLLKSYEGGRESHAHFIAKSLFEEYLMVEELFSDGIQSDVIETLRHQHSKDLQKVVDIVLSHQGVRNKAKLVTALMEKLVYPNPVAYRDLLVRFSSLNHKRYYKLALKASELLEQTKLSELRASIARSLSDLGMHKGDMTIKDSMEDLVSAPLPVEDALISLFDYSDATVQQKVIETYISRLYQPHLVKDSIQMKFKEYGAIAFWEFSEGHVDTSNGHGTILGGKRWGSMVVLKSLESASTAIVAALKDSAQYNSSEGNTMHIALLSAENESNMSGTSSDDQAQHRMEKLTKILKDTNVASDLRAAGLKVISCIVQRDEARMPMRHTFLWLDEKSCYEEEQILRHVEPPLSALLELDKLKVKGYNEMKYTPSRDRQWHIYTLRNTENPKMLHRVFFRTIVRQPNAGNKFTSAQVSDTEVGGPEDSLSFTSNSILRSLMTAIEELELHAIRTGHSHMYLCILKEQKLLDLIPFSGSTIVDVGQDEATACSLLKSMALKIHDLVGARMHHLSVCQWEVKLKLDCDGPASGTWRVVTTNVTSHTCTIDIYREVEDTESQKLVYHSTTSAAGPGPLHGVALNNPYQPLSVIDLKRCSARNNRTTYCYDFPLAFETALQKSWQSNGSSVSVGSGNSKSYVKATELVFAEKHGSWGTPIVPMERPAGLNDIGMVAWILEMSTPEFPNGRQIIVVANDITFRAGSFGPREDAFFEAVTNLACERKLPLIYLAANSGARIGIADEVKSCFRVGWSDEGSPERGFQYIYLTEEDYARISSSVIAHKLQLDSGEIRWIIDSVVGKEDGLGVENIHGSAAIASAYSRAYEETFTLTFVTGRTVGIGAYLARLGIRCIQRLDQPIILTGFSALNKLLGREVYSSHMQLGGPKIMATNGVVHLTVPDDLEGVSNILRWLSYVPANIGGPLPITKPLDPPDRPVAYIPENTCDPRAAIRGVDDSQGKWLGGMFDKDSFVETFEGWAKTVVTGRAKLGGIPVGVIAVETQTMMQLVPADPGQLDSHERSVPRAGQVWFPDSATKTAQALLDFNREGLPLFILANWRGFSGGQRDLFEGILQAGSTIVENLRTYNQPAFVYIPMAGELRGGAWVVVDSKINPDRIECYAERTAKGNVLEPQGLIEIKFRSEELQDCMGRLDPELINLKAKLQDVKHGNGSLPDIESLQKSIEARTKQLLPLYTQIAIRFAELHDTSLRMAAKGVIKKVVDWEESRSFFYKRLRRRISEDVLAKEIRHIVGDNFTHQSAMELIKEWYLASPATAGSTGWDDDDAFVAWKDSPENYNGYIQELRAQKVSQSLSDLTDSSSDLQAFSQGLSTLLDKMDPSQRAKFVQEVKKVLG

SEQ ID NO: 3 - DNA, 6984 bp, Sorghum bicolor
  source 1..6984: mol_type = genomic DNA
atgtcgcagcttggattagctgcagctgcctcaaaggcactgccactactccctaatcgccagcgaagtcctgctgggactacattctcatcatctgcattgtcgaggccctcaaaccgaaggaaaagccgtacccgttcactccgtgatggtggagatggggtatcagatgtcaaaaagcacagccagtctgttcgtcaaggtcttgctggcattattgacctcccaagcgaggcagcttcggaagtggacatttcacatggatctgaggatcctaggggtccaccagattcctatcaaatgaatgggattatcaatgaaacacataatggaagacatgcttcagtgtccaaggttgttgaattttgtgcggcactaggtggcaaaacaccaattcacagtatattagtggccaataatggaatggcagcagcaaagttcatgaggagtgtccggacatgggctaatgatacttttggatctgagaaggcaattcagctcatagctatggcaactccggaagacatgaggataaatgcagagcacattagaattgctgatcaatttgtagaggtgcctggtggaacaaacaataataactacgccaacgttcaactcatagtggaggtagcagaaagagtaggtgtttctgctgtttggcctggttggggtcatgcttctgagaatcctgaactgccggatgcattgactgcaaaaggaatcgttttccttgggccacctgcatcatcaatgaatgcattgggagataaggtcggttcagctctcattgctcaagcagctggggtcccaactcttgcttggagtggatcacatgttgaagtgccattagagtgctgcttagacgctatacctgaggagatgtatagaaaagcttgtgttactaccacagaggaagcagttgcaagttgtcaagtgattggttatcctgccatgattaaggcatcctggggaggtggtggtaaaggaataagaaaggttcataatgatgatgaggttagagcactgtttaagcaagtacaaggtgaagtccctggctccccaatatttatcatgaggcttgcagcccagagtcggcatcttgaagttcagttgctttgtgatcaatatggcaatgtagcagcacttcacagtcgtgattgcagtgtgcaacggcgacaccagaagattattgaagaaggcccagttactgttgctcctcgtgagacagttaaagcacttgagcaggcagcaaggaggcttgctaaggctgtgggttatgttggtgctgctactgttgagtatctttacagcatggaaactggagaatactattttctggagcttaatccccgactacaggtcgagcatccagtcaccgaatggatagctgaagtaaatctgcctgcagctcaagttgctgttggaatgggcatacctctttggcaaattccagaaatcagacgtttctatggaatggactatggaggagggtatgatatttggaggaaaacagcagctcttgctacaccatttaattttgatgaagtagattctcaatggccaaagggccattgtgtagcagttagaattactagtgaggaccctgatgatggtttcaaacctactggtgggaaagtgaaggagataacttttaaagccaagcctaatgtttgggcctacttctcagtaaagtctggtggaggcattcatgaatttgctgattctcagtttggacatgtttttgcatatggactctctagatcagcagcaataacaaacatggctcttgcattaaaagagattcaaattcgtggagaaattcattcaaatgttgattacactgttgacctcttaaacgcttcagacttcagagaaaacaagattcatactggttggctggataccagaatagctatgcgtgttcaagctgagaggcccccatggtatatttcagtggttggaggtgctttatacaaaacagtaaccaccaatgcagccactgtttctgactatgttagttatctcaccaagggccagattccaccaaagcatatatcccttgtcaattctacagttagcttgaatatagaagggagcaaatacacaattgaaactgtaaggactggacatggtagctacaggttgcgaatgaatgaatcgacagttgaagcgaatgtacaatctttatgtgatggtggactcttaatgcagttggatggaaacagccatgtaatttatgcagaagaagaagctggtggtacacggcttcagattgatggaaagacatgcttgttgcagaatgaccatgatccatcaaagttattagctgagacaccctgcaaacttcttcgtttcttggttgctgatggtgctcatgttgatgcggatgtaccatacgctgaagttgaggttatgaagatgtgcatgcctctcctgtcacctgcttctggtgtcgttcattgtatgatgtctgagggccaggcgttgcaggctggtgatcttatagcaaggctggatcttgatgacccttctgctgtgaaaagggctgaaccatttgatggaatatttccacaaatggcgctccctgttgctgcctctagtcaagtacacaaaagatatgctgcaagtttgaatgctgctcgaatggttcttgcaggatatgagcacaatatcaatgaagtcgttcaagatttgatatgctgcctggacaaccctgagcttcctttcctacagtgggatgaacttatgtctgttctagcaacgaggcttccaagaaatctcaagagtgagttagaggataaatacaaggaatacaagttgaatttttaccgtggaaaaaacgtggacttcccttccaagttgctaagagacatcattgaggaaaatcttgcatatggttcagagaaggaaaaggctacaaatgagaggcttgttgagcctcttacgaacctactgaagtcatatgagggtgggagagaaagtcatgcacattttattgccaagtcccttttcgaggagtatcttatggtggaagaacttttcagtgatggaattcagtctgacgttattgaaaccttgcgtcatcagcacagtaaagacctgcagaaggttgtagacattgtgttgtctcaccagggtgtgagaaacaaagctaagcttgtaacagcactcatggaaaagctggtttatccaaatccggttgcttacagggatctgttggttcgcttttcttccctcaatcataaaagatattataagttggcccttaaagcaagtgaacttcttgaacaaaccaaactaagtgaactccgtgcaagcattgcaagaagcctttcagatctggggatgcataagggagacatgactattaaggatagcatggaagatttagtctctgccccattgcctgttgaagatgctcttatttctttgtttgattacagcgatgcaactgttcagcagaaagtgattgagacatacatatcacgattgtaccagcctcatcttgtgaaggatagcatccaaatgaaattcaaggaatatggtgctattgctttttgggaattttctgaagggcatgttgatacttcaaatggacatgggactattcttggtgggaagagatggggttccatggtcgtcctcaaatcacttgaatctgcatcaacagccattgtggctgcattaaaggattcagcacagtacaacagctctgagggcaacacgatgcacattgcattattgagtgctgaaaatgaaagtaatatgagtggaacaagcagtgatgatcaagctcaacataggatggaaaagcttaccaagatactgaaggatactaatgttgcaagtgatctccgagctgctggtttgaaggttataagttgcattgttcaaagagatgaagctcgcatgccaatgcgccacacattcctctggttggatgaaaagagttgttatgaagaagagcaaattctccggcatgtggagcctcccctctctgcacttcttgaattggataagttgaaagtgaaaggatacaatgaaatgaagtatactccgtcacgtgatcgtcaatggcatatctacacactaagaaatactgaaaaccccaaaatgttgcatagggtatttttccgaactattgtcaggcaacccaatgcaggcaacaagtttacatcagctcaggtcagtgatactgaagtaggaggtcctgaagattctctgtcattcacatcgaatagcatcctaagatcattgatgactgctatagaagaattagagcttcatgcgattaggacaggtcattctcacatgtatttgtgcatactgaaagagcaaaagcttcttgatctcattccattttcagggagtacaattgttgatgttggccaagatgaagctactgcttgttcacttttaaaatcaatggctttgaagatacatgaccttgttggtgcaaggatgcatcatctgtctgtatgccagtgggaggtgaaactcaagttggattgtgatgggcctgcaagtggtacctggagagttgtgactacaaatgttactagtcacacctgcaccattgatatctaccgagaagtggaagatacagaatcgcagaagttagtataccattcaaccacttcggcagctggtcctggtccgttgcatggtgttgcactgaataatccatatcaacctttaagtgtgattgatctaaagcgctgctctgctaggaacaacagaacaacatattgctacgattttccgctggcatttgaaactgcactgcagaagtcatggcagtccaacggctctagtgtttctgtaggcagtggaaatagtaaatcctacgtgaaggcaactgagctggtgtttgctgaaaaacatgggtcctggggcactcctatagttcccatggaacgtcctgctgggctcaatgacattggtatggtcgcttggatcttggagatgtcaacacctgaatttcccaatggcaggcagattattgttgtagcaaatgatattactttcagagctggatcattcggcccaagggaagatgcattttttgaagctgtcaccaacctggcttgtgaaaggaaacttccccttatatacttggcagcaaactctggtgctaggattggcatagctgatgaagtaaaatcttgcttccgtgttgggtggtctgacgaaggcagccctgagcgagggtttcagtacatctatctgactgaagaagactatgcccgtattagctcttctgttatagcacataagctgcagctagatagcggtgaaattaggtggattattgactctgttgtgggcaaggaggatgggcttggtgttgagaacatacatggaagtgctgctatcgccagtgcttattctagggcatatgaggagacatttacacttacatttgtgaccggacggactgtaggaataggagcttatcttgctagacttggtatacggtgcatacagcgtcttgaccagccgattattttaacagggttttctgccctgaacaagctccttgggcgggaagtgtacagctcccacatgcagcttggtggtcctaagatcatggcgaccaatggtgttgtccacctgactgttccagatgaccttgaaggtgtttccaatatattgaggtggctcagctatgttcctgcaaacattggtggacctcttcctattaccaaacctttggaccctccagacagacctgttgcatacatccctgagaacacatgcgatccacgtgcagccatccgtggtgtagatgacagccaagggaaatggttgggtggtatgtttgacaaagacagctttgtggagacatttgaaggatgggcaaaaacagtggttactggcagagcaaagcttggaggaattcctgtgggtgtcatagctgtggagacacagaccatgatgcagcttgtccctgctgatccaggtcagcttgattcccatgagcgatccgttcctcgggctggacaagtgtggttcccagattctgcaaccaagacagctcaggcattattagacttcaaccgtgaaggattgcctctgtttatcctggctaactgcagaggtttctctggtggacagagagatctctttgaaggaattcttcaggctgggtcaacaattgtcgagaaccttaggacatataatcagcctgcgtttgtctacattcctatggctggagagcttcgtggaggagcttgggttgtggtcgatagcaaaataaatccagaccgcattgagtgttatgctgagaggactgccaaaggtaatgttctcgaacctcaagggttaattgaaatcaagttcaggtcagaggaactccaagactgtatgggtaggcttgaccccgagttgataaatctgaaagcaaaactccaagatgtaaagcatggaaatggaagtctaccagacatagaatcccttcagaagagtatagaagcacgtacgaaacagttgctgcctttatatacccagattgcaatacggtttgctgaattgcatgatacttccctaagaatggcagctaaaggcgtgattaagaaagttgtagactgggaagaatcacgctctttcttctataaaaggctacggagaaggatctctgaagatgttcttgcaaaagaaataagacatatagtcggtgacaacttcactcaccaatcagcaatggagctcatcaaggaatggtacctggcttctccagccacagcaggaagcactggatgggatgacgatgatgcatttgttgcctggaaggacagtcctgaaaactacaatggatatatccaagagctaagggctcaaaaagtgtctcagtcgctctctgatctcactgactccagttcagatctacaagcattctcgcagggtctttctacgctattagataagatggatccctctcaaagagcgaagtttgttcaggaagtcaagaaggtccttggttga

SEQ ID NO: 4 - protein, 2327 aa, Sorghum bicolor
MSQLGLAAAASKALPLLPNRQRSPAGTTFSSSALSRPSNRRKSRTRSLRDGGDGVSDVKKHSQSVRQGLAGIIDLPSEAASEVDISHGSEDPRGPPDSYQMNGIINETHNGRHASVSKVVEFCAALGGKTPIHSILVANNGMAAAKFMRSVRTWANDTFGSEKAIQLIAMATPEDMRINAEHIRIADQFVEVPGGTNNNNYANVQLIVEVAERVGVSAVWPGWGHASENPELPDALTAKGIVFLGPPASSMNALGDKVGSALIAQAAGVPTLAWSGSHVEVPLECCLDAIPEEMYRKACVTTTEEAVASCQVIGYPAMIKASWGGGGKGIRKVHNDDEVRALFKQVQGEVPGSPIFIMRLAAQSRHLEVQLLCDQYGNVAALHSRDCSVQRRHQKIIEEGPVTVAPRETVKALEQAARRLAKAVGYVGAATVEYLYSMETGEYYFLELNPRLQVEHPVTEWIAEVNLPAAQVAVGMGIPLWQIPEIRRFYGMDYGGGYDIWRKTAALATPFNFDEVDSQWPKGHCVAVRITSEDPDDGFKPTGGKVKEITFKAKPNVWAYFSVKSGGGIHEFADSQFGHVFAYGLSRSAAITNMALALKEIQIRGEIHSNVDYTVDLLNASDFRENKIHTGWLDTRIAMRVQAERPPWYISVVGGALYKTVTTNAATVSDYVSYLTKGQIPPKHISLVNSTVSLNIEGSKYTIETVRTGHGSYRLRMNESTVEANVQSLCDGGLLMQLDGNSHVIYAEEEAGGTRLQIDGKTCLLQNDHDPSKLLAETPCKLLRFLVADGAHVDADVPYAEVEVMKMCMPLLSPASGVVHCMMSEGQALQAGDLIARLDLDDPSAVKRAEPFDGIFPQMALPVAASSQVHKRYAASLNAARMVLAGYEHNINEVVQDLICCLDNPELPFLQWDELMSVLATRLPRNLKSELEDKYKEYKLNFYRGKNVDFPSKLLRDIIEENLAYGSEKEKATNERLVEPLTNLLKSYEGGRESHAHFIAKSLFEEYLMVEELFSDGIQSDVIETLRHQHSKDLQKVVDIVLSHQGVRNKAKLVTALMEKLVYPNPVAYRDLLVRFSSLNHKRYYKLALKASELLEQTKLSELRASIARSLSDLGMHKGDMTIKDSMEDLVSAPLPVEDALISLFDYSDATVQQKVIETYISRLYQPHLVKDSIQMKFKEYGAIAFWEFSEGHVDTSNGHGTILGGKRWGSMVVLKSLESASTAIVAALKDSAQYNSSEGNTMHIALLSAENESNMSGTSSDDQAQHRMEKLTKILKDTNVASDLRAAGLKVISCIVQRDEARMPMRHTFLWLDEKSCYEEEQILRHVEPPLSALLELDKLKVKGYNEMKYTPSRDRQWHIYTLRNTENPKMLHRVFFRTIVRQPNAGNKFTSAQVSDTEVGGPEDSLSFTSNSILRSLMTAIEELELHAIRTGHSHMYLCILKEQKLLDLIPFSGSTIVDVGQDEATACSLLKSMALKIHDLVGARMHHLSVCQWEVKLKLDCDGPASGTWRVVTTNVTSHTCTIDIYREVEDTESQKLVYHSTTSAAGPGPLHGVALNNPYQPLSVIDLKRCSARNNRTTYCYDFPLAFETALQKSWQSNGSSVSVGSGNSKSYVKATELVFAEKHGSWGTPIVPMERPAGLNDIGMVAWILEMSTPEFPNGRQIIVVANDITFRAGSFGPREDAFFEAVTNLACERKLPLIYLAANSGARIGIADEVKSCFRVGWSDEGSPERGFQYIYLTEEDYARISSSVIAHKLQLDSGEIRWIIDSVVGKEDGLGVENIHGSAAIASAYSRAYEETFTLTFVTGRTVGIGAYLARLGIRCIQRLDQPIILTGFSALNKLLGREVYSSHMQLGGPKIMATNGVVHLTVPDDLEGVSNILRWLSYVPANIGGPLPITKPLDPPDRPVAYIPENTCDPRAAIRGVDDSQGKWLGGMFDKDSFVETFEGWAKTVVTGRAKLGGIPVGVIAVETQTMMQLVPADPGQLDSHERSVPRAGQVWFPDSATKTAQALLDFNREGLPLFILANCRGFSGGQRDLFEGILQAGSTIVENLRTYNQPAFVYIPMAGELRGGAWVVVDSKINPDRIECYAERTAKGNVLEPQGLIEIKFRSEELQDCMGRLDPELINLKAKLQDVKHGNGSLPDIESLQKSIEARTKQLLPLYTQIAIRFAELHDTSLRMAAKGVIKKVVDWEESRSFFYKRLRRRISEDVLAKEIRHIVGDNFTHQSAMELIKEWYLASPATAGSTGWDDDDAFVAWKDSPENYNGYIQELRAQKVSQSLSDLTDSSSDLQAFSQGLSTLLDKMDPSQRAKFVQEVKKVLG

SEQ ID NO: 5 - DNA, 24 bp, Sorghum bicolor
  source 1..24: mol_type = genomic DNA
cctctgtttatcctggctaactgg

SEQ ID NO: 6 - DNA, 23 bp, Sorghum bicolor
  source 1..23: mol_type = genomic DNA
aggattgcctctgtttatcctgg

SEQ ID NO: 7 - DNA, 23 bp, Sorghum bicolor
  source 1..23: mol_type = genomic DNA
tttatcctggctaactggagagg

SEQ ID NO: 8 - DNA, 20 bp, Synthetic construct
  source 1..20: mol_type = other DNA
gtctgtttatcctggctaac

SEQ ID NO: 9 - DNA, 20 bp, Synthetic construct
  source 1..20: mol_type = other DNA
gggattgcctctgtttatcc

SEQ ID NO: 10 - DNA, 20 bp, Synthetic construct
  source 1..20: mol_type = other DNA
gttatcctggctaactggag

SEQ ID NO: 11 - DNA, 20 bp, Synthetic construct
  source 1..20: mol_type = other DNA
gtatcctggctaactgtgag

SEQ ID NO: 12 - DNA, 23 bp, Synthetic construct
  source 1..23: mol_type = other DNA
ttatcctggctaactgtgagagg

SEQ ID NO: 13 - DNA, 77 bp, Synthetic construct
  source 1..77: mol_type = other DNA
gttttagagctagaaatagcaagttaaaataaggctagtccgttatcaacttgaaaaagtggcaccgagtcggtgct

SEQ ID NO: 14 - DNA, 204 bp, Synthetic construct
  source 1..204: mol_type = other DNA
gggctggacaagtgtggttcccagattctgcaaccaagacagctcaggcattattagacttcaaccgtgaaggattgcctctgtttatcctggctaactgcagaggtttctctggtggacagagagatctctttgaaggaattcttcaggctgggtcaacaattgtcgagaaccttaggacatataatcagcctgcgtttgtct

SEQ ID NO: 15 - DNA, 35 bp, Synthetic construct
  source 1..35: mol_type = other DNA
atcgggaagctgaagtctgcaaccaagacagctca

SEQ ID NO: 16 - DNA, 35 bp, Synthetic construct
  source 1..35: mol_type = other DNA
atccgacggtagtgtgacccagcctgaagaattcc

SEQ ID NO: 17 - DNA, 34 bp, Synthetic construct
  source 1..34: mol_type = other DNA
atcgggaagctgaagatgagcgatccgttcctcg

SEQ ID NO: 18 - DNA, 35 bp, Synthetic construct
  source 1..35: mol_type = other DNA
atccgacggtagtgtgaccacaacccaagctcctc

SEQ ID NO: 19 - DNA, 1000 bp, Synthetic construct
  source 1..1000: mol_type = other DNA
tgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaatt

(19) United States
(12) Patent Application Publication
(10) Pub. No.: US 2026/0234651 A1
(43) Pub. Date: Aug. 13, 2026

(54) ACETYL-COA CARBOXYLASE INHIBITOR RESISTANT SORGHUM

(71) Applicant: PIONEER HI-BRED INTERNATIONAL, INC., JOHNSTON, IA (US)

(72) Inventors: HUIRONG GAO, JOHNSTON, IA (US); LEANDRO DANIEL PERUGINI, URBANDALE, IA (US)

(73) Assignee: PIONEER HI-BRED INTERNATIONAL, INC., JOHNSTON, IA (US)

(21) Appl. No.: 19/469,649

(22) PCT Filed: Mar. 28, 2024
(86) PCT No.: PCT/US2024/021864

§ 371 Date: Sep. 26, 2025

Related U.S. Application Data
(60) Provisional application No. 63/492,864, filed on Mar. 29, 2023.

(51) Int. Cl. C12N 15/82; C12N 9/22; C12N 15/11

(52) U.S. Cl. CPC C12N 15/8274; C12N 9/226; C12N 15/111; C12N 15/8213; C12N 15/8275

(57) ABSTRACT

Disclosed herein are Sorghum plants and seeds thereof having an introduced genetic modification that results in improved tolerance to acetyl-CoA carboxylase (ACCase) inhibiting herbicides. Also disclosed herein are methods to produce the Sorghum plants and methods for controlling weeds in a field by growing the Sorghum plants and treating with an ACCase inhibitor herbicide.

### REFERENCE TO SEQUENCE LISTING SUBMITTED ELECTRONICALLY

The official copy of the sequence listing is submitted electronically via Patent Center as an XML formatted sequence listing with a file named 9389_SequenceListing created on Feb. 21, 2023 and having a size of 46,282 bytes and is filed concurrently with the specification. The sequence listing comprised in this XML formatted document is part of the specification and is herein incorporated by reference in its entirety.

### BACKGROUND

The development of herbicide resistance in plants offers significant production and economic advantages; as such the use of herbicides for controlling weeds or plants in crops has become almost a universal practice. Of particular interest to farmers is the use of herbicides with high potency, broad weed spectrum effectiveness and rapid soil degradation. One such class of broad-spectrum herbicides are those compounds that inhibit the activity of the acetyl-CoA carboxylase (ACCase) enzyme in a plant. Such herbicides are included in the aryloxyphenoxypropionate (FOP) and cyclohexanedione (DIM) chemical families.

Sorghum is susceptible to many ACCase inhibiting herbicides that target monocot species, making the use of these herbicides to control grassy weeds in Sorghum fields almost impossible. The development of Sorghum varieties that are resistant to the inhibitory effects of ACCase inhibiting herbicides would allow for greater crop yield when these herbicides are used to control grassy weeds. Therefore, due to the importance of Sorghum as a crop plant on the world stage, Sorghum varieties that are resistant to the inhibitory effects of ACCase inhibiting herbicides along with methods for producing and using said hybrids are needed.

### SUMMARY

Provided is a genome edited Sorghum variety comprising an introduced genetic modification at an endogenous acetyl-CoA carboxylase (ACCase) gene to encode a modified ACCase polypeptide that is at least 90% identical to SEQ ID NO: 2 and comprises a non-tryptophan (e.g., arginine, histidine, lysine, aspartic acid, glutamic acid, serine, threonine, asparagine, glutamine, glycine, proline, cysteine, alanine, valine, isoleucine, leucine, methionine, phenylalanine, or tyrosine) at a position corresponding to position 2032 of SEQ ID NO: 2. In certain embodiments, the modified ACCase polypeptide is at least 90% identical to SEQ ID NO: 2 and comprises a cysteine at the position corresponding to position 2032 of SEQ ID NO: 2. In certain embodiments, the edited Sorghum variety has increased tolerance to at least one ACCase inhibitor herbicide as compared to a control Sorghum variety not comprising the introduced genetic modification.

Also provided is an edited Sorghum hybrid comprising an introduced genetic modification at an endogenous ACCase gene to encode a modified ACCase polypeptide that is at least 90% identical to SEQ ID NO: 2 and comprises a non-tryptophan (e.g., arginine, histidine, lysine, aspartic acid, glutamic acid, serine, threonine, asparagine, glutamine, glycine, proline, cysteine, alanine, valine, isoleucine, leucine, methionine, phenylalanine, or tyrosine) at a position corresponding to position 2032 of SEQ ID NO: 2. In certain embodiments, the modified ACCase polypeptide is at least 90% identical to SEQ ID NO: 2 and comprises a cysteine at the position corresponding to position 2032 of SEQ ID NO: 2. In certain embodiments, the edited Sorghum hybrid has increased tolerance to at least one ACCase inhibitor herbicide as compared to a control Sorghum hybrid not comprising the introduced genetic modification.

Provided is a method for producing an ACCase inhibitor tolerant Sorghum hybrid, the method comprising crossing a first Sorghum variety comprising an introduced genetic modification at an ACCase gene to encode a modified ACCase polypeptide that is at least 90% identical to SEQ ID NO: 2 and comprises a non-tryptophan at a position corresponding to position 2032 of SEQ ID NO: 2, with a second Sorghum variety. In certain embodiments, the modified ACCase polypeptide of the first Sorghum variety is at least 90% identical to SEQ ID NO: 2 and comprises a cysteine at the position corresponding to position 2032 of SEQ ID NO: 2. In certain embodiments, the second Sorghum variety comprises an introduced genetic modification at an ACCase gene to encode a modified ACCase polypeptide that is at least 90% identical to SEQ ID NO: 2 and comprises a non-tryptophan at a position corresponding to position 2032 of SEQ ID NO: 2 at one allele. In certain embodiments, the modified ACCase polypeptide of the second Sorghum variety is at least 90% identical to SEQ ID NO: 2 and comprises a cysteine at the position corresponding to position 2032 of SEQ ID NO: 2.

Also provided is a method for producing an ACCase inhibitor tolerant Sorghum variety comprising introducing into a regenerable Sorghum plant cell a genetic modification at an endogenous ACCase gene to encode a modified ACCase polypeptide that is at least 90% identical to SEQ ID NO: 2 and comprises a cysteine at a position corresponding to position 2032 of SEQ ID NO: 2, and generating a Sorghum plant from the plant cell, the generated plant comprising the introduced genetic modification and has improved tolerance to ACCase inhibitor herbicides as compared to a control plant.

Further provided is a method for producing an ACCase inhibitor tolerant Sorghum variety comprising providing to a plant cell comprising an ACCase gene sequence a guide RNA, a polynucleotide modification template comprising at least one nucleotide modification of the ACCase gene sequence to encode a modified ACCase polypeptide that is at least 90% identical to SEQ ID NO: 2 and comprises a cysteine at a position corresponding to position 2032 of SEQ ID NO: 2, and a Cas endonuclease, the guide RNA and Cas endonuclease capable of forming a complex that enables the Cas endonuclease to introduce a double strand break at a target site sequence in the ACCase gene of said plant cell, obtaining a plant from the plant cell, evaluating the plant for the presence of said at least one nucleotide modification, and selecting a progeny Sorghum plant comprising the modified ACCase gene and having increased tolerance to ACCase inhibitor herbicides as compared to a control plant not comprising the modification. In certain embodiments, the method further comprises crossing the selected progeny Sorghum plant with a second Sorghum plant to produce an F1 progeny plant.

Provided is a method for introgressing an modified ACCase allele into an elite Sorghum variety comprising crossing a first Sorghum variety with a second Sorghum variety to produce a progeny population, the first Sorghum variety comprising an introduced genetic modification at an endogenous ACCase gene to encode a modified ACCase polypeptide that is at least 90% identical to SEQ ID NO: 2 and comprises a non-tryptophan at a position corresponding to position 2032 of SEQ ID NO: 2, genotyping the progeny population for the presence of the introduced genetic modification, and selecting progeny that compare the introduced genetic modification to obtain soybean plants comprising the modified ACCase allele and having improved tolerance to ACCase inhibitor herbicides. In certain embodiments, the modified ACCase polypeptide of the first Sorghum variety is at least 90% identical to SEQ ID NO: 2 and comprises a cysteine at the position corresponding to position 2032 of SEQ ID NO: 2. In certain embodiments, the second Sorghum variety comprises an introduced genetic modification at an ACCase gene to encode a modified ACCase polypeptide that is at least 90% identical to SEQ ID NO: 2 and comprises a non-tryptophan at a position corresponding to position 2032 of SEQ ID NO: 2 at one allele. In certain embodiments, the modified ACCase polypeptide of the second Sorghum variety is at least 90% identical to SEQ ID NO: 2 and comprises a cysteine at the position corresponding to position 2032 of SEQ ID NO: 2.

Also provided is a method for controlling weeds in an area of cultivation comprising planting an area of cultivation with seeds and/or plants comprising an introduced genetic modification at an ACCase gene to encode a modified ACCase polypeptide that is at least 90% identical to SEQ ID NO: 2 and comprises a non-tryptophan at a position corresponding to position 2032 of SEQ ID NO: 2 and applying to the seeds and/or plants a sufficient amount of an ACCase inhibitor herbicide to control the weeds without significantly affecting the seeds and/or plants. In certain embodiments, the modified ACCase polypeptide is at least 90% identical to SEQ ID NO: 2 and comprises a cysteine at a position corresponding to position 2032 of SEQ ID NO: 2.

### BRIEF DESCRIPTION OF THE DRAWING AND THE SEQUENCE LISTING

The disclosure can be more fully understood from the following detailed description and the accompanying drawing and Sequence Listing, which form a part of this application.

The sequence descriptions (Table 1) and sequence listing attached hereto comply with the rules governing nucleotide and amino acid sequence disclosures in patent applications as set forth in 37 C.F.R. §§ 1.831-1.835.

FIG. 1 provides the target site location on the ACCase gene sequence (SEQ ID NO: 1) for ACC-TS1, ACC-TS2, and ACC-TS3. The nucleotide sequence shown corresponds to positions 14411 to 14458 of SEQ ID NO: 1. The location of the nucleotide sequence to modify is highlighted in bold.

TABLE 1
Sequence Listing Description

| SEQ ID NOS | Description |
|---|---|
| 1 | Wild-type ACCase - genomic sequence |
| 2 | Wild-type ACCase - amino acid sequence |
| 3 | Genome edited ACCase - genomic sequence |
| 4 | Genome edited ACCase - amino acid sequence |
| 5 | ACC-TS1 |
| 6 | ACC-TS2 |
| 7 | ACC-TS3 |
| 8 | ACC-CR1 |
| 9 | ACC-CR2 |
| 10 | ACC-CR3 |
| 11 | ACC-CR3.1 |
| 12 | ACC-TS3.1 |
| 13 | Guide RNA |
| 14 | Polynucleotide Modification Template |
| 15 | ACC-TS3F - Primer |
| 16 | ACC-TS3R - Primer |
| 17 | ACC-editF - Primer |
| 18 | ACC-editR - Primer |
| 19 | Zm-U6 Pol III promoter |

### DETAILED DESCRIPTION

Acetyl-CoA carboxylase (ACCase) is a biotinylated enzyme that catalyzes the carboxylation of acetyl-CoA to produce malonyl-CoA. This carboxylation is a two-step, reversible reaction consisting of the ATP-dependent carboxylation of the biotin group on the carboxyl carrier domain by biotin-carboxylase activity followed by the transfer of the carboxyl group from biotin to acetyl-CoA by carboxyl-transferase activity (Nikolau et al., 2003, Arch. Biochem. Biophys. 414:211-22). Acetyl-CoA carboxylase is not only a key enzyme in plants for biosynthesis of fatty acids, a process that occurs in chloroplasts and mitochondria, but ACCase also plays a role in the formation of long-chain fatty acids and flavonoids, and in malonylation that occurs in the cytoplasm. There are two isoforms of ACCase with the chloroplastic ACCase accounting for more than 80% of the total ACCase activity (Herbert et al., 1996, Biochem. J. 318:997-1006). Aryloxyphenoxypropionate (FOP) and cyclohexanedione (DIM) are two classes of chemicals that are known to selectively inhibit chloroplastic ACCase in grasses (Rendina et al., 1990, J. Agric. Food Chem. 38:1282-1287).

Cultivated Sorghum [Sorghum bicolor (L.) Moench] is susceptible to many acetyl-CoA carboxylase (ACCase) inhibiting herbicides that target monocot or grassy weed species, which greatly limits the use of these herbicides to control grassy weeds in Sorghum fields. Thus, the development of Sorghum varieties that are resistant to the inhibitory effects of ACCase inhibiting herbicides would allow for greater crop yield when these herbicides are used to control grassy weeds. Accordingly, the present disclosure provides genome edited Sorghum varieties having increased resistance to ACCase inhibitor herbicides as compared to a control Sorghum variety not comprising the genome edit, and methods for producing and using the genome edited varieties.

Provided herein are genome edited Sorghum varieties and seeds producing the genome edited Sorghum varieties comprising an introduced genetic modification at an endogenous acetyl-CoA carboxylase (ACCase) gene to encode a modified ACCase polypeptide that is at least, or at least about, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprises a non-tryptophan (e.g., arginine, histidine, lysine, aspartic acid, glutamic acid, serine, threonine, asparagine, glutamine, glycine, proline, cysteine, alanine, valine, isoleucine, leucine, methionine, phenylalanine, or tyrosine) at a position corresponding to position 2032 of SEQ ID NO: 2. In certain embodiments the genome edited Sorghum varieties and seeds producing the genome edited Sorghum varieties comprise an introduced genetic modification at an endogenous acetyl-CoA carboxylase (ACCase) gene to encode a modified ACCase polypeptide that is at least or at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprises a cysteine at a position corresponding to position 2032 of SEQ ID NO: 2. In certain embodiments, the endogenous ACCase gene comprises a polynucleotide sequence that is at least or at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1. In certain embodiments, the modified ACCase polypeptide comprises the amino acid sequence of SEQ ID NO: 4. In certain embodiments, the genome edited Sorghum variety has increased tolerance to at least one (e.g., 1, 2, 3, 4, 5, 6, 7, 8, 9, 10, or more) ACCase inhibitor herbicide as compared to a control Sorghum variety not comprising the introduced genetic modification, such as, for example a Sorghum variety comprising an ACCase gene encoding an ACCase polypeptide comprising SEQ ID NO: 2. In certain embodiments, the genome edited Sorghum variety has increased tolerance to at least one (e.g., 1, 2, 3, 4, 5, 6, 7, 8, 9, 10, or more) ACCase inhibitor herbicide as compared to a control Sorghum variety comprising an ACCase gene encoding an ACCase polypeptide comprising a cysteine at a position corresponding to position 2032 of SEQ ID NO: 2 introduced by crossing with an exotic Sorghum variety.

As used herein, “gene” includes a nucleic acid fragment that expresses a functional molecule such as, but not limited to, a specific protein coding sequence and regulatory elements, such as those preceding (5′ non-coding sequences) and following (3′ non-coding sequences) the coding sequence.

The terms “polypeptide,” “peptide” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers.

As used herein “encoding,” “encoded,” or the like, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons.

Typically, the amino acid sequence is encoded by the nucleic acid using the “universal” genetic code. However, variants of the universal code, such as is present in some plant, animal and fungal mitochondria, the bacterium Mycoplasma capricolum (Yamao, et al., (1985) Proc. Natl. Acad. Sci. USA 82:2306-9) or the ciliate Macronucleus, may be used when the nucleic acid is expressed using these organisms.

As used herein “percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100).

Unless otherwise stated, sequence identity/similarity values provided herein refer to the value obtained using the BLAST 2.0 suite of programs using default parameters (Altschul, et al., (1997) Nucleic Acids Res. 25:3389-402).

The genetic modification of the endogenous ACCase gene may be introduced using any genome modification technique known in the art or described herein. In certain embodiments the targeted DNA modification is through a genome modification technique using an enzyme selected from the group consisting of a polynucleotide-guided endonuclease, CRISPR-Cas endonucleases, base editing deaminases, zinc finger nuclease, a transcription activator-like effector nuclease (TALEN) or engineered site-specific meganuclease.

In certain embodiments, the genome modification may be facilitated through the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), guided cpf1 endonuclease systems, Cas12f endonuclease systems and the like. In certain embodiments, the introduction of a DSB can be combined with the introduction of a polynucleotide modification template.

As used herein, the term “variety” includes whole plants, plant organs, plant tissues, seeds, plant cells, seeds, grain and progeny of the same.

In certain embodiments, the genome edit is introduced into an elite Sorghum variety thereby producing a genome edited elite Sorghum variety. As used herein, an “elite line” “elite variety” or the like is an agronomically superior line that has resulted from many cycles of breeding and selection for superior agronomic performance. Numerous elite lines are available and known to those of skill in the art of Sorghum breeding. As used herein, an “exotic Sorghum line” “exotic Sorghum variety” or the like is a strain or germplasm derived from a Sorghum not belonging to an available elite Sorghum line or strain of germplasm. In the context of a cross between two Sorghum plants or strains of germplasm, an exotic germplasm is not closely related by descent to the elite germplasm with which it is crossed. Most commonly, the exotic germplasm is not derived from any known elite line of soybean, but rather is selected to introduce novel genetic elements (typically novel alleles) into a breeding program.

In certain embodiments, the elite variety is an elite inbred variety. As used herein, an “elite inbred” refers to an elite line or variety that has been bred for genetic homogeneity. In certain embodiments, the elite variety is an elite hybrid variety. As used herein, an “elite hybrid” refers to the progeny obtained between the crossing of at least two genetically dissimilar parents, such as, for example, two elite inbred parents. Methods to develop elite Sorghum inbred and hybrid lines are known in the art, and numerous elite lines are available and known to those of skill in the art of Sorghum breeding.

As used herein, the term “germplasm” refers to genetic material of or from an individual (e.g., a plant), a group of individuals (e.g., a plant line, variety or family), or a clone derived from a line, variety, species, or culture. The germplasm can be part of an organism or cell or can be separate from the organism or cell. The germplasm provides genetic material with a specific molecular makeup that provides a physical foundation for some or all of the hereditary qualities of an organism or cell culture. Germplasm in the context of the present disclosure includes cells, seed or tissues from which new plants can be grown, or plant parts, such as leaves, stems, pollen, or cells, that can be cultured into a whole plant.

In certain embodiments, the genome edited variety (e.g., elite inbred variety and/or elite hybrid variety) has a yield that is greater than, equal to, or within 0.5%, 1%, 1.5%, 2%, 2.5%, 3%, 3.5%, 4%, 4.5%, or 5%, as compared to the corresponding control plant, for example, one which has a similar genetic background but lacks the introduced genetic modification. In certain embodiments, the genome edited variety (e.g., elite inbred variety and/or elite hybrid variety) has a yield that is increased by at least about 0.5%, 1%, 1.5%, 2%, 2.5%, 3%, 3.5%, 4%, 4.5%, or 5%, as compared to a control Sorghum variety comprising an ACCase gene encoding an ACCase polypeptide comprising a cysteine at a position corresponding to position 2032 of SEQ ID NO: 2 introduced by crossing with an exotic Sorghum variety.

In certain embodiments, the genome edited Sorghum variety (e.g., elite inbred variety and/or elite hybrid variety) has a yield that is increased by at least about 0.5%, 1%, 1.5%, 2%, 2.5%, 3%, 3.5%, 4%, 4.5%, 5%, 10% or more when grown in the presence of an ACCase inhibitor herbicide, as compared to a control Sorghum variety comprising an ACCase gene encoding an ACCase polypeptide comprising a cysteine at a position corresponding to position 2032 of SEQ ID NO: 2 introduced by crossing with an exotic Sorghum variety. In certain embodiments, the genome edited elite hybrid variety has a yield of at least 80 bushels per acre (bu/A), 85 bu/A, 90 bu/A, 95 bu/A, 100 bu/A, 105 bu/A, 110 bu/A, 115 bu/A, 120 bu/A, 125 bu/A, 130 bu/A, 140 bu/A, 150 bu/A, 175 bu/A, or 200 bu/A when grown in the presence of at least one ACCase inhibitor herbicide. In certain embodiments, the at least one ACCase inhibitor is selected from the aryloxyphenoxypropionate (FOP) herbicide family or the cyclohexanediones (DIM) herbicide family. In certain embodiments, the at least one ACCase inhibitor is quizalofop-p-ethyl. In certain embodiments, the ACCase inhibitor herbicide, such as, for example, quizalofop-p-ethyl is applied at a rate of at least about 0.04 pounds active ingredient per acre (lb ai/A), 0.045 lb ai/A, 0.05 lb ai/A, 0.055 lb ai/A, 0.06 lb ai/A, 0.065 lb ai/A, 0.07 lb ai/A, 0.075 lb ai/A, 0.08 lb ai/A, 0.085 lb ai/A, 0.09 lb ai/A, 0.095 lb ai/A, 0.1 lb ai/A, 0.15 lb ai/A, 0.2 lb ai/A, or 0.25 lb ai/A and less than 0.3 lb ai/A, 0.25 lb ai/A, 0.2 lb ai/A, 0.15 lb ai/A, 0.1 lb ai/A, 0.095 lb ai/A, 0.09 lb ai/A, 0.085 lb ai/A, 0.08 lb ai/A, 0.075 lb ai/A, 0.07 lb ai/A, or 0.06 lb ai/A. In certain embodiments, the at least one ACCase inhibitor is quizalofop-p-ethyl applied at a rate ranging from about 0.05 lb ai/A to 0.15 lb ai/A.

As used herein, “yield” refers to the amount of agricultural production harvested per unit of land and may include reference to bushels per acre or kilograms per hectare of a crop at harvest, as adjusted for grain moisture. Grain moisture is measured in the grain at harvest. The adjusted test weight of grain is determined to be the weight in pounds per bushel or kilogram, adjusted for grain moisture level at harvest.

In certain embodiments of the genome edited Sorghum elite inbred varieties and/or elite hybrid varieties or seeds thereof described herein, the introduced genetic modification is present at a single allele of the ACCase gene, such that the genome edited Sorghum elite inbred variety and/or elite hybrid variety is heterozygous for the introduced genetic modification. In certain embodiments of the genome edited Sorghum elite inbred varieties and/or elite hybrid varieties or seeds thereof described herein, the introduced genetic modification is present at a both alleles of the ACCase gene, such that the genome edited Sorghum elite inbred variety and/or elite hybrid variety is homozygous for the introduced genetic modification.

In certain embodiments, the genome edited Sorghum elite inbred varieties and/or elite hybrid varieties described herein have tolerance to at least one aryloxyphenoxypropionate (FOP) herbicide family member. In certain embodiments, the genome edited Sorghum elite inbred varieties and/or elite hybrid varieties described herein have tolerance to quizalofop-p-ethyl when applied at a rate of at least at least about 0.04 lb ai/A, 0.045 lb ai/A, 0.05 lb ai/A, 0.055 lb ai/A, 0.06 lb ai/A, 0.065 lb ai/A, 0.07 lb ai/A, 0.075 lb ai/A, 0.08 lb ai/A, 0.085 lb ai/A, 0.09 lb ai/A, 0.095 lb ai/A, 0.1 lb ai/A, 0.15 lb ai/A, 0.2 lb ai/A, or 0.25 lb ai/A and less than 0.3 lb ai/A, 0.25 lb ai/A, 0.2 lb ai/A, 0.15 lb ai/A, 0.1 lb ai/A, 0.095 lb ai/A, 0.09 lb ai/A, 0.085 lb ai/A, 0.08 lb ai/A, 0.075 lb ai/A, 0.07 lb ai/A, or 0.06 lb ai/A. In certain embodiments, the quizalofop-p-ethyl is applied at a rate ranging from about 0.05 lb ai/A to 0.15 lb ai/A. In certain embodiments, the genome edited Sorghum elite inbred varieties and/or elite hybrid varieties described herein have tolerance to at least one cyclohexanediones (DIM) herbicide family member. In certain embodiments, the genome edited Sorghum elite inbred varieties and/or elite hybrid varieties described herein have tolerance to clethodim when applied at a rate of at least 0.04 lb ai/A, 0.045 lb ai/A, 0.05 lb ai/A, 0.055 lb ai/A, 0.06 lb ai/A, 0.065 lb ai/A, 0.07 lb ai/A, 0.075 lb ai/A, 0.08 lb ai/A, 0.085 lb ai/A, 0.09 lb ai/A, 0.095 lb ai/A, 0.1 lb ai/A, 0.15 lb ai/A, 0.2 lb ai/A, or 0.25 lb ai/A and less than 0.3 lb ai/A, 0.25 lb ai/A, 0.2 lb ai/A, 0.15 lb ai/A, 0.1 lb ai/A, 0.095 lb ai/A, 0.09 lb ai/A, 0.085 lb ai/A, 0.08 lb ai/A, 0.075 lb ai/A, 0.07 lb ai/A, or 0.06 lb ai/A. In certain embodiments, the clethodim is applied at a rate ranging from about 0.08 lb ai/A to 0.2 lb ai/A. In certain embodiments, the genome edited Sorghum elite inbred varieties and/or elite hybrid varieties described herein have tolerance to a combination herbicide comprising at least one aryloxyphenoxypropionate (FOP) herbicide family member and at least one cyclohexanediones (DIM) herbicide family member. Members of the FOP herbicide family are known in the art and include, but not limited to, clodinafop-propargyl, cyhalofop-butyl, diclofop-methyl, fenoxaprop-p-ethyl, fluazifop-b-butyl, haloxyfop-ethoxyethyl, haloxyfop-etotyl, haloxyfop-R-methyl, propaquizafop, quizalofop-p-ethyl and quizalo-P-refuryl compounds. Similarly, members of the DIM herbicide family are known in the art and include, but not limited to, alloxydim, butroxydim, clefoxydim, clethodim, cycloxydim, profoxydim, sethoxydim, tepraloxydim and tralkoxydim compounds.

As used herein, “herbicide tolerance” “herbicide resistance” and the like refers to plants that exhibit few, if any, bleached, necrotic, lytic, chlorotic or other lesions and are not stunted, wilted or deformed when subjected to the herbicide at concentrations and rates which are typically employed by the agricultural community to kill weeds in the field. As used herein, “increased tolerance” “increased resistance” or the like refers to any detectable decrease in bleached, necrotic, lytic, chlorotic or other lesions or any detectable decrease in number of stunted, wilted or deformed plants as compared to an appropriate control when subjected to the herbicide. When comparing two plants the plant that exhibits the least amount of bleached, necrotic, lytic, chlorotic or other lesions or least amount of stunted, wilted or deformed plants will be considered to have increased tolerance as compared to the other plant. Increased tolerance can be demonstrated when plants which display the increased tolerance to a herbicide are subjected to the ACCase inhibitor and a dose/response curve is shifted to the right when compared with that provided by an appropriate control plant.

In certain embodiments, the genome edited Sorghum varieties (e.g., elite inbred varieties and/or elite hybrid varieties) and seeds thereof described herein can further comprise one or more additional genetic modifications of the endogenous ACCase gene that are associated with tolerance or increased tolerance to ACCase inhibitor herbicides. In certain embodiments, the genome edited Sorghum varieties (e.g., elite inbred varieties and/or elite hybrid varieties) and seeds thereof described herein can further comprise one or more additional genetic modifications of the endogenous ACCase gene to increase expression and/or activity of the encoded polypeptide. In certain embodiments, the one or more additional genetic modifications of the endogenous ACCase gene comprise a modification of a regulatory element. In certain embodiments, the regulatory element is the endogenous ACCase gene promoter. In certain embodiments, the endogenous ACCase gene promoter is swapped for a promoter sequence that results in higher expression of the ACCase gene. In certain embodiments, the one or more additional genetic modifications of the endogenous ACCase gene are introduced at the same time as the edit resulting in the W2032C mutation in the encoded polypeptide.

A “regulatory sequence” generally refers to a transcriptional regulatory element involved in regulating the transcription of a nucleic acid molecule such as a gene or a target gene. The regulatory element is a nucleic acid and may include a promoter, an enhancer, an intron, a 5′-untranslated region (5′-UTR, also known as a leader sequence), or a 3′-UTR or a combination thereof. A “promoter” refers to a region of DNA upstream from the start of transcription and involved in recognition and binding of RNA polymerase and other proteins to initiate transcription. An “enhancer” element is any nucleic acid molecule that increases transcription of a nucleic acid molecule when functionally linked to a promoter regardless of its relative position. An “intron” is an intervening sequence in a gene that is transcribed into RNA but is then excised in the process of generating the mature mRNA. The term is also used for the excised RNA sequences. The 5′ untranslated region (5′UTR) (also known as a translational leader sequence or leader RNA) is the region of an mRNA that is directly upstream from the initiation codon. This region is involved in the regulation of translation of a transcript by differing mechanisms in viruses, prokaryotes and eukaryotes. The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

In certain embodiments, the endogenous ACCase gene promoter is swapped with a heterologous promoter with a constitutive promoter or a tissue-preferred promoter.

Also provided herein are genome edited Sorghum inbred and hybrid varieties and seeds thereof having increased tolerance to ACCase inhibitor herbicides and comprising a genetic modification introducing a modified acetyl-CoA carboxylase (ACCase) gene that encodes a modified ACCase polypeptide that is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprises a non-tryptophan (e.g., a cysteine) at a position corresponding to position 2032 of SEQ ID NO: 2, the modified ACCase gene being introduced at genomic locus other than the endogenous ACCase gene locus. In certain embodiments, the modified ACCase gene is operably linked to a heterologous regulatory element, such as, for example, a heterologous promoter. In certain embodiments, the inbred and hybrid varieties are elite inbred and hybrid varieties.

In certain embodiments, the genome edited Sorghum varieties (e.g., elite inbred varieties and/or elite hybrid varieties) and seeds thereof described herein can further comprise one or more additional traits of interest. A trait, as used herein, refers to the phenotype derived from a particular sequence or groups of sequences. In certain embodiments, the genome edited Sorghum varieties and seeds thereof comprise at least one additional polynucleotide that confers tolerance to at least one additional herbicide, such as, for example one or more sequences that confer tolerance to: an ALS inhibitor; an HPPD inhibitor; 2,4-D; other phenoxy auxin herbicides; glyphosate; dicamba; glufosinate herbicides; herbicides which target the protox enzyme (also referred to as “protox inhibitors”). In certain embodiments, the genome edited Sorghum varieties (e.g., elite inbred varieties and/or elite hybrid varieties) and seeds thereof described herein further comprise at least one additional polynucleotide that confers tolerance to a herbicide. In certain embodiments, the at least one additional polynucleotide that confers tolerance to Glyphosate, 2,4-D, Glufosinate, or any combination thereof. In certain embodiments, the at least one additional polynucleotide that confers tolerance to Glyphosate, 2,4-D, and Glufosinate. In certain embodiments, the genome edited Sorghum varieties (e.g., elite inbred varieties and/or elite hybrid varieties) and seeds thereof described herein further comprise a polynucleotide that confers tolerance to Glyphosate, a polynucleotide that confers tolerance to 2,4-D, and a polynucleotide that confers tolerance to Glufosinate. The at least one additional polynucleotide can be introduced into the Sorghum variety using any method known in the art, such as, for example, backcrossing, transformation, genome editing, or locus conversion.

The genome edited Sorghum varieties (e.g., elite inbred varieties and/or elite hybrid varieties) and seeds thereof described herein can also be combined with at least one other trait to produce plants that further comprise a variety of desired trait combinations. For instance, the genome edited Sorghum variety may be stacked with polynucleotides encoding polypeptides having pesticidal and/or insecticidal activity, or a plant, plant cell, plant part, seed, and/or grain comprising the genome modification provided herein may be combined with a plant disease resistance gene.

In certain embodiments, the genome edited Sorghum variety comprises at least one additional polynucleotide that confers increased seed protein or oil content. For instance, a modified polynucleotide encoding a diacylglycerol acyltransferase (DGAT) polypeptide, such as those described in WO19/232182, or a high oleic acid trait, such as those described in U.S. Pat. No. 8,609,935.

These combinations can be created by any method including, but not limited to, breeding plants by any conventional methodology, or genetic transformation. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853, all of which are herein incorporated by reference.

Also provided is a method for producing an ACCase inhibitor tolerant Sorghum hybrid comprising crossing a first Sorghum inbred variety comprising any Sorghum inbred variety described herein with a second inbred Sorghum variety. In certain embodiments, the method further comprises collecting the seeds from a progeny population produced from the cross and growing the collected seeds. In certain embodiments, the first inbred Sorghum variety comprises an introduced genetic modification at an endogenous acetyl-CoA carboxylase (ACCase) gene to encode a modified ACCase polypeptide that is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprises a non-tryptophan (e.g., a cysteine) at a position corresponding to position 2032 of SEQ ID NO: 2. In certain embodiments, the non-tryptophan is a cysteine such that the modified ACCase polypeptide comprises a cysteine at the position corresponding to position 2032 of SEQ ID NO: 2. In certain embodiments, the introduced genetic modification is present on a single allele of the ACCase gene in the first Sorghum variety. In certain embodiments, the introduced genetic modification is present on a both alleles of the ACCase gene in the first Sorghum variety. In certain embodiments, the second inbred Sorghum variety also comprises an introduced genetic modification at an endogenous acetyl-CoA carboxylase (ACCase) gene to encode a modified ACCase polypeptide that is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprises a non-tryptophan (e.g., a cysteine) at a position corresponding to position 2032 of SEQ ID NO: 2. In certain embodiments, the non-tryptophan is a cysteine such that the modified ACCase polypeptide in the second Sorghum variety comprises a cysteine at the position corresponding to position 2032 of SEQ ID NO: 2. In certain embodiments, the introduced genetic modification is present on a single allele of the ACCase gene in the second Sorghum variety. In certain embodiments, the introduced genetic modification is present on a both alleles of the ACCase gene in the second Sorghum variety. In certain embodiments, the second inbred Sorghum variety does not comprise the introduced genetic modification.

In certain embodiments, the produced hybrid is heterozygous for the modified ACCase gene. In certain embodiments, the produced hybrid is homozygous for the modified ACCase gene. In certain embodiments, the produced hybrid has tolerance to at least one aryloxyphenoxypropionate (FOP) herbicide family member. In certain embodiments, the produced hybrid has tolerance to quizalofop-p-ethyl when applied at a rate of at least or at least about 0.04 lb ai/A, 0.045 lb ai/A, 0.05 lb ai/A, 0.055 lb ai/A, 0.06 lb ai/A, 0.065 lb ai/A, 0.07 lb ai/A, 0.075 lb ai/A, 0.08 lb ai/A, 0.085 lb ai/A, 0.09 lb ai/A, 0.095 lb ai/A, 0.1 lb ai/A, 0.15 lb ai/A, 0.2 lb ai/A, or 0.25 lb ai/A and less than 0.3 lb ai/A, 0.25 lb ai/A, 0.2 lb ai/A, 0.15 lb ai/A, 0.1 lb ai/A, 0.095 lb ai/A, 0.09 lb ai/A, 0.085 lb ai/A, 0.08 lb ai/A, 0.075 lb ai/A, 0.07 lb ai/A, or 0.06 lb ai/A. In certain embodiments, the quizalofop-p-ethyl is applied at a rate ranging from about 0.05 lb ai/A to 0.15 lb ai/A. In certain embodiments, the produced hybrid has tolerance to at least one cyclohexanedione (DIM) herbicide family member. In certain embodiments, the produced hybrid has tolerance to clethodim when applied at a rate of at least 0.04 lb ai/A, 0.045 lb ai/A, 0.05 lb ai/A, 0.055 lb ai/A, 0.06 lb ai/A, 0.065 lb ai/A, 0.07 lb ai/A, 0.075 lb ai/A, 0.08 lb ai/A, 0.085 lb ai/A, 0.09 lb ai/A, 0.095 lb ai/A, 0.1 lb ai/A, 0.15 lb ai/A, 0.2 lb ai/A, or 0.25 lb ai/A and less than 0.3 lb ai/A, 0.25 lb ai/A, 0.2 lb ai/A, 0.15 lb ai/A, 0.1 lb ai/A, 0.095 lb ai/A, 0.09 lb ai/A, 0.085 lb ai/A, 0.08 lb ai/A, 0.075 lb ai/A, 0.07 lb ai/A, or 0.06 lb ai/A. In certain embodiments, the clethodim is applied at a rate ranging from about 0.08 lb ai/A to 0.2 lb ai/A. In certain embodiments, the produced hybrid has tolerance to a combination herbicide comprising at least one aryloxyphenoxypropionate (FOP) herbicide family member and at least one cyclohexanedione (DIM) herbicide family member. In certain embodiments, the produced hybrid has a yield that is greater than, equal to, or within 0.5%, 1%, 1.5%, 2%, 2.5%, 3%, 3.5%, 4%, 4.5%, or 5%, as compared to the corresponding control plant, for example, one which has a similar genetic background but lacks the introduced genetic modification. In certain embodiments, the produced hybrid has a yield that is increased by at least about 0.5%, 1%, 1.5%, 2%, 2.5%, 3%, 3.5%, 4%, 4.5%, or 5%, as compared to a control Sorghum variety comprising an ACCase gene encoding an ACCase polypeptide comprising a cysteine at a position corresponding to position 2032 of SEQ ID NO: 2 introduced by crossing with an exotic Sorghum variety.

Sorghum varieties are mainly self-pollinated; therefore, self-pollination of the parental varieties must be controlled to make hybrid variety development feasible. A pollination control system and effective transfer of pollen from one parent to the other offers improved plant breeding and an effective method for producing hybrid seed and plants. For example, the milo or A₁ cytoplasmic male sterility (CMS) system, developed via a cross between milo and kafir cultivars, is one of the most frequently used CMS systems in hybrid Sorghum production (Stephens J C & Holland P F, Cytoplasmic Male Sterility for Hybrid Sorghum Seed Production, Agron. J. 46:20-23 (1954)). Other CMS systems for Sorghum include, but are not limited to, A₂, isolated from IS 12662c (Schertz K F, Registration of A₂Tₓ 2753 and BTₓ 2753 Sorghum Germplasm, Crop Sci. 17:983 (1977)), A₃, isolated from IS 1112c or converted Nilwa (Quinby J R, Interactions of Genes and Cytoplasms in Male-Sterility in Sorghums, Proc. 35th Corn Sorghum Res. Conf. Am. Seed Trade Assoc. Chicago, Ill., pp. 5-8 (1980)), A₄, isolated from IS 7920c (Worstell et al, Relationship among Male-Sterility Inducing Cytoplasms of Sorghum, Crop Sci. 24:186-189 (1984)).

In developing improved new Sorghum varieties, breeders may use a CMS plant as the female parent. In using these plants, breeders attempt to improve the efficiency of seed production and the quality of the F₁ hybrids and to reduce the breeding costs. When hybridization is conducted without using CMS plants, it is more difficult to obtain and isolate the desired traits in the progeny (F₁ generation) because the parents are capable of undergoing both cross-pollination and self-pollination. If one of the parents is a CMS plant that is incapable of producing pollen, only cross pollination will occur. By eliminating the pollen of one parental variety in a cross, a plant breeder is assured of obtaining seed of uniform quality, provided that the parents are of uniform quality and the breeder conducts a single cross.

In certain embodiments, production of hybrid varieties described herein includes crossing a CMS female parent with a pollen-producing male parent. In certain embodiments, the CMS female parent comprises any of the genetic modifications of the ACCase gene described herein. In certain embodiments, the pollen-producing male parent comprises any of the genetic modifications of the ACCase gene described herein. In certain embodiments, both the CMS female parent and the pollen-producing male parent comprise any of the genetic modifications of the ACCase gene described herein. To reproduce effectively, however, the male parent of the F₁ hybrid has a fertility restorer gene (Rf gene). The presence of an Rf gene means that the F₁ generation will not be completely or partially sterile, so that either self-pollination or cross pollination may occur. Self-pollination of the F₁ generation to produce several subsequent generations ensures that a desired trait is heritable and stable and that a new variety has been isolated.

Further provided herein are methods for producing an ACCase inhibitor tolerant Sorghum variety comprising introducing into a regenerable Sorghum plant cell a genetic modification at an endogenous acetyl-CoA carboxylase (ACCase) gene to encode a modified ACCase polypeptide that is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprises a non-tryptophan (e.g., a cysteine) at a position corresponding to position 2032 of SEQ ID NO: 2, and generating a Sorghum plant from the plant cell, the generated plant comprising the introduced genetic modification and has improved tolerance to ACCase inhibitor herbicides as compared to a control plant. In certain embodiments, the non-tryptophan is a cysteine such that the modified ACCase polypeptide comprises a cysteine at the position corresponding to position 2032 of SEQ ID NO: 2. In certain embodiments, the genetic modification is introduced using a genome modification technique selected from the group consisting of a polynucleotide-guided endonuclease, CRISPR-Cas endonucleases, base editing deaminases, a zinc finger nuclease, a transcription activator-like effector nuclease (TALEN) and engineered site-specific endonucleases.

In certain embodiments, the genome edit may be facilitated through the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, CRISPR-Cas systems (e.g., Cas9-gRNA systems, guided cpf1 endonuclease systems, Cas12f endonuclease systems and the like). In some embodiments, the introduction of a DSB can be combined with the introduction of a polynucleotide modification template.

The process for editing a genomic sequence combining DSB and modification templates generally comprises providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

The endonuclease can be provided to a cell by any method known in the art, for example, but not limited to, transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433.

TAL effector nucleases (TALEN) are a class of sequence-specific nucleases that can be used to make double-strand breaks at specific target sequences in the genome of a plant or other organism (Miller et al. (2011) Nature Biotechnology 29:143-148).

Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US12/30061). Meganucleases have been classified into four families based on conserved sequence motifs. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. The cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a non-specific endonuclease domain, for example nuclease domain from a Type IIs endonuclease such as FokI. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3-finger domain recognized a sequence of 9 contiguous nucleotides, with a dimerization requirement of the nuclease, two sets of zinc finger triplets are used to bind an 18-nucleotide recognition sequence.

Genome editing using DSB-inducing agents, such as Cas9-gRNA complexes, has been described, for example in U.S. Patent Application US 2015-0082478 A1, WO2015/026886 A1, WO2016007347, and WO201625131 all of which are incorporated by reference herein.

In certain embodiments the genetic modification is introduced without introducing a double strand break using base editing technology, see e.g., Gaudelli et al., (2017) Programmable base editing of A*T to G*C in genomic DNA without DNA cleavage. Nature 551 (7681): 464-471; Komor et al., (2016) Programmable editing of a target base in genomic DNA without double-stranded DNA cleavage, Nature 533 (7603): 420-4.

In certain embodiments, base editing comprises (i) a catalytically impaired CRISPR-Cas9 mutant that is mutated such that one of their nuclease domains cannot make DSBs; (ii) a single-strand-specific cytidine/adenine deaminase that converts C to U or A to G within an appropriate nucleotide window in the single-stranded DNA bubble created by Cas9; (iii) a uracil glycosylase inhibitor (UGI) that impedes uracil excision and downstream processes that decrease base editing efficiency and product purity; or (iv) nickase activity to cleave the non-edited DNA strand, followed by cellular DNA repair processes to replace the G-containing DNA strand.

Also provided is a method for producing an ACCase inhibitor tolerant Sorghum variety comprising providing to a Sorghum plant cell comprising an endogenous ACCase gene sequence a guide polynucleotide, a polynucleotide modification template comprising at least one nucleotide modification of the ACCase gene sequence to encode a modified ACCase polypeptide that is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprises a cysteine at a position corresponding to position 2032 of SEQ ID NO: 2, and a Cas endonuclease, the guide polynucleotide and Cas endonuclease capable of forming a complex that enables the Cas endonuclease to introduce a double strand break at a target site sequence in the ACCase gene of said plant cell, obtaining a plant from the plant cell, evaluating the plant for the presence of said at least one nucleotide modification, and selecting a progeny Sorghum plant comprising the modified ACCase gene and having increased tolerance to ACCase inhibitor herbicides as compared to a control plant not comprising the modification.

As used herein, the term “guide polynucleotide”, relates to a polynucleotide sequence that can form a complex with a Cas endonuclease, including the Cas endonuclease described herein, and enables the Cas endonuclease to recognize, optionally bind to, and optionally cleave a DNA target site. The guide polynucleotide sequence can be an RNA sequence, a DNA sequence, or a combination thereof (a RNA-DNA combination sequence). In certain embodiments, the guide polynucleotide is a guide RNA (gRNA) comprising a CRISPR RNA (crRNA) and a trans-activating CRISPR RNA (tracrRNA) to guide the Cas endonuclease to its DNA target. The crRNA comprises a spacer region complementary to one strand of the double strand DNA target and a region that base pairs with the tracrRNA, forming an RNA duplex. In certain embodiments, the gRNA is a “single guide RNA” (sgRNA) that comprises a synthetic fusion of crRNA and tracrRNA.

In certain embodiments of the methods described herein, the guide RNA comprises a crRNA-tracrRNA fusion transcript. In certain embodiments, the tracrRNA comprises a nucleotide sequence that is at least 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 13. In certain embodiments, the crRNA comprises a nucleotide sequence that is at least 75%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 8, 9, or 10.

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited. In certain embodiments of the methods described herein, the polynucleotide modification template comprises a nucleotide sequence that is at least 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 14.

The terms “target site”, “target sequence”, “target site sequence, “target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, a locus, or any other DNA molecule in the genome (including chromosomal, chloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell. In certain embodiments, the target site sequence in the endogenous ACCase gene comprises a nucleotide sequence that is at least 75%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 5, 6, 7, or 12.

The Cas endonuclease for use in the methods described herein is not particularly limited and may be any Cas endonuclease known in the art. In certain embodiments, the Cas endonuclease is a Class 2 Type II. In Class 2 Type II systems, the Cas endonuclease acts in complex with a guide RNA (gRNA) that directs the Cas endonuclease to cleave the DNA target to enable target recognition, binding, and cleavage by the Cas endonuclease. The gRNA comprises a Cas endonuclease recognition (CER) domain that interacts with the Cas endonuclease, and a Variable Targeting (VT) domain that hybridizes to a nucleotide sequence in a target DNA. In certain embodiments, the Cas endonuclease is selected from the group consisting of Cas9, Cpf1 and Cas12f (also known in the art as Cas-alpha 10).

In certain embodiments, the progeny plant comprises the modification on a single allele of the ACCase gene. In certain embodiments, the progeny plant comprises the modification on both alleles of the ACCase gene. In certain embodiments, the progeny plant has tolerance to at least one aryloxyphenoxypropionate (FOP) herbicide family member. In certain embodiments, the progeny plant has tolerance to quizalofop-p-ethyl when applied at a rate of at least or at least about 0.04 lb ai/A, 0.045 lb ai/A, 0.05 lb ai/A, 0.055 lb ai/A, 0.06 lb ai/A, 0.065 lb ai/A, 0.07 lb ai/A, 0.075 lb ai/A, 0.08 lb ai/A, 0.085 lb ai/A, 0.09 lb ai/A, 0.095 lb ai/A, 0.1 lb ai/A, 0.15 lb ai/A, 0.2 lb ai/A, or 0.25 lb ai/A and less than 0.3 lb ai/A, 0.25 lb ai/A, 0.2 lb ai/A, 0.15 lb ai/A, 0.1 lb ai/A, 0.095 lb ai/A, 0.09 lb ai/A, 0.085 lb ai/A, 0.08 lb ai/A, 0.075 lb ai/A, 0.07 lb ai/A, or 0.06 lb ai/A. In certain embodiments, the quizalofop-p-ethyl is applied at a rate ranging from about 0.05 lb ai/A to 0.15 lb ai/A. In certain embodiments, the progeny plant has tolerance to at least one cyclohexanedione (DIM) herbicide family member. In certain embodiments, the progeny plant has tolerance to clethodim when applied at a rate of at least 0.04 lb ai/A, 0.045 lb ai/A, 0.05 lb ai/A, 0.055 lb ai/A, 0.06 lb ai/A, 0.065 lb ai/A, 0.07 lb ai/A, 0.075 lb ai/A, 0.08 lb ai/A, 0.085 lb ai/A, 0.09 lb ai/A, 0.095 lb ai/A, 0.1 lb ai/A, 0.15 lb ai/A, 0.2 lb ai/A, or 0.25 lb ai/A and less than 0.3 lb ai/A, 0.25 lb ai/A, 0.2 lb ai/A, 0.15 lb ai/A, 0.1 lb ai/A, 0.095 lb ai/A, 0.09 lb ai/A, 0.085 lb ai/A, 0.08 lb ai/A, 0.075 lb ai/A, 0.07 lb ai/A, or 0.06 lb ai/A. In certain embodiments, the clethodim is applied at a rate ranging from about 0.08 lb ai/A to 0.2 lb ai/A. In certain embodiments, the progeny plant has tolerance to a combination herbicide comprising at least one aryloxyphenoxypropionate (FOP) herbicide family member and at least one cyclohexanedione (DIM) herbicide family member. In certain embodiments, the progeny plant has a yield that is greater than, equal to, or within 0.5%, 1%, 1.5%, 2%, 2.5%, 3%, 3.5%, 4%, 4.5%, or 5%, as compared to the corresponding control plant, for example, one which has a similar genetic background but lacks the introduced genetic modification. In certain embodiments, the progeny plant has a yield that is increased by at least about 0.5%, 1%, 1.5%, 2%, 2.5%, 3%, 3.5%, 4%, 4.5%, or 5%, as compared to a control Sorghum variety comprising an ACCase gene encoding an ACCase polypeptide comprising a cysteine at a position corresponding to position 2032 of SEQ ID NO: 2 introduced by crossing with an exotic Sorghum variety.

In certain embodiments, the method further comprises crossing the selected progeny Sorghum plant with a second Sorghum plant to produce an F1 hybrid progeny plant. In certain embodiments, the second Sorghum plant comprises an ACCase polynucleotide encoding a modified ACCase polypeptide that is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprises a cysteine at a position corresponding to position 2032 of SEQ ID NO: 2. In certain embodiments, the F1 hybrid progeny plant is homozygous for the modified ACCase polynucleotide. In certain embodiments, the F1 hybrid progeny plant is heterozygous for the modified ACCase polynucleotide. In certain embodiments, the F1 hybrid progeny plant has tolerance to at least one aryloxyphenoxypropionate (FOP) herbicide family member. In certain embodiments, the F1 hybrid progeny plant has tolerance to quizalofop-p-ethyl when applied at a rate of at least or at least about 0.04 lb ai/A, 0.045 lb ai/A, 0.05 lb ai/A, 0.055 lb ai/A, 0.06 lb ai/A, 0.065 lb ai/A, 0.07 lb ai/A, 0.075 lb ai/A, 0.08 lb ai/A, 0.085 lb ai/A, 0.09 lb ai/A, 0.095 lb ai/A, 0.1 lb ai/A, 0.15 lb ai/A, 0.2 lb ai/A, or 0.25 lb ai/A and less than 0.3 lb ai/A, 0.25 lb ai/A, 0.2 lb ai/A, 0.15 lb ai/A, 0.1 lb ai/A, 0.095 lb ai/A, 0.09 lb ai/A, 0.085 lb ai/A, 0.08 lb ai/A, 0.075 lb ai/A, 0.07 lb ai/A, or 0.06 lb ai/A. In certain embodiments, the quizalofop-p-ethyl is applied at a rate ranging from about 0.05 lb ai/A to 0.15 lb ai/A. In certain embodiments, the F1 hybrid progeny plant has tolerance to at least one cyclohexanedione (DIM) herbicide family member. In certain embodiments, the F1 hybrid progeny plant has tolerance to clethodim when applied at a rate of at least 0.04 lb ai/A, 0.045 lb ai/A, 0.05 lb ai/A, 0.055 lb ai/A, 0.06 lb ai/A, 0.065 lb ai/A, 0.07 lb ai/A, 0.075 lb ai/A, 0.08 lb ai/A, 0.085 lb ai/A, 0.09 lb ai/A, 0.095 lb ai/A, 0.1 lb ai/A, 0.15 lb ai/A, 0.2 lb ai/A, or 0.25 lb ai/A and less than 0.3 lb ai/A, 0.25 lb ai/A, 0.2 lb ai/A, 0.15 lb ai/A, 0.1 lb ai/A, 0.095 lb ai/A, 0.09 lb ai/A, 0.085 lb ai/A, 0.08 lb ai/A, 0.075 lb ai/A, 0.07 lb ai/A, or 0.06 lb ai/A. In certain embodiments, the clethodim is applied at a rate ranging from about 0.08 lb ai/A to 0.2 lb ai/A. In certain embodiments, the F1 hybrid progeny plant has tolerance to a combination herbicide comprising at least one aryloxyphenoxypropionate (FOP) herbicide family member and at least one cyclohexanedione (DIM) herbicide family member. In certain embodiments, the F1 hybrid progeny plant has a yield that is greater than, equal to, or within 0.5%, 1%, 1.5%, 2%, 2.5%, 3%, 3.5%, 4%, 4.5%, or 5%, as compared to the corresponding control plant, for example, one which has a similar genetic background but lacks the introduced genetic modification. In certain embodiments, the F1 hybrid progeny plant has a yield that is increased by at least about 0.5%, 1%, 1.5%, 2%, 2.5%, 3%, 3.5%, 4%, 4.5%, or 5%, as compared to a control Sorghum variety comprising an ACCase gene encoding an ACCase polypeptide comprising a cysteine at a position corresponding to position 2032 of SEQ ID NO: 2 introduced by crossing with an exotic Sorghum variety.

Also provided is a method for introgressing a modified ACCase allele into an elite Sorghum variety comprising crossing a first Sorghum variety with a second Sorghum variety to produce a progeny population, the first Sorghum variety comprising any of the introduced genetic modifications described herein, genotyping the progeny population for the presence of the introduced genetic modification, and selecting progeny that comprise the introduced genetic modification to obtain Sorghum varieties comprising the modified ACCase allele and having improved tolerance to ACCase inhibitor herbicides. In certain embodiments, the selected progeny comprises a single ACCase allele having the introduced genetic modification. In certain embodiments, the selected progeny comprises the introduced genetic modification at both alleles. In certain embodiments, the presence of the introduced genetic modification is detected using primers selected from the group consisting of SEQ ID NOs: 15, 16, 17, and 18.

Also provided is a method for controlling and/or selectively controlling weeds in an area of cultivation comprising planting an area of cultivation with seeds and/or plants comprising any introduced genetic modifications described herein and applying to the seeds and/or plants a sufficient amount of an ACCase inhibitor herbicide to control the weeds without significantly affecting the seeds and/or plants. In certain embodiments, the seed produces an elite Sorghum hybrid variety comprising an introduced genetic modification at endogenous acetyl-CoA carboxylase (ACCase) gene to encode a modified ACCase polypeptide that is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 and comprises a non-tryptophan (e.g., a cysteine) at a position corresponding to position 2032 of SEQ ID NO: 2. In certain embodiments, the non-tryptophan is a cysteine such that the modified ACCase polypeptide comprises a cysteine at the position corresponding to position 2032 of SEQ ID NO: 2.

Weed” as used herein refers to a plant which is not desirable in a particular area. In certain embodiments, a weed is a non-crop plant or a non-crop species, while in some embodiments, a weed is a crop species which is sought to be eliminated from a particular area.

As used herein the term “controlling,” and derivations thereof, for example, as in “controlling weeds” refers to one or more of inhibiting the growth, germination, reproduction, and/or proliferation of; and/or killing, removing, destroying, or otherwise diminishing the occurrence and/or activity of a weed. As used herein, by “selectively controlled” it is intended that the majority of weeds in an area of cultivation are significantly damaged or killed, while if crop plants are also present in the field, the majority of the crop plants are not significantly damaged. Thus, a method is considered to selectively control weeds when at least 55%, 60%, 65%, 70%, 75%, 80%, 85%, 90%, 95%, or more of the weeds are significantly damaged or killed, while if crop plants are also present in the field, less than 45%, 40%, 35%, 30%, 25%, 20%, 15%, 10%, 5%, or 1% of the crop plants are significantly damaged or killed.

As used herein, an “area of cultivation” comprises any region in which one desires to grow a plant. Such areas of cultivations include, but are not limited to, a field in which a plant is cultivated, a greenhouse, or a growth chamber.

In certain embodiments, the ACCase inhibitor herbicide is applied in a pre-emergence application. In certain embodiments, the ACCase inhibitor herbicide is applied in a post-emergence application. In certain embodiments, the ACCase inhibitor herbicide is applied in both a pre-emergence and post-emergence application. In certain embodiments, a non-ACCase inhibitor herbicide (e.g., glyphosate, 2,4-D, glufosinate) is applied in a pre-emergence application and the ACCase inhibitor herbicide is applied in a post-emergence application.

When the Sorghum variety further comprises at least one additional polynucleotide that confers tolerance to a herbicide (e.g., glyphosate, 2,4-D, glufosinate) the herbicide may be applied in a pre-emergence application, a post-emergence application, or both a pre-emergence and post-emergence application while the ACCase inhibitor herbicide may be simultaneously or sequentially applied in a pre-emergence application, a post-emergence application, or both a pre-emergence and post-emergence application.

“Pre-emergent” “pre-emergence” or the like refers to a herbicide which is applied to an area of interest (e.g., a field or area of cultivation) before a plant emerges visibly from the soil. “Post-emergent” “post-emergence” or the like refers to a herbicide which is applied to an area after a plant emerges visibly from the soil. In some instances, the terms “pre-emergent” and “post-emergent” are used with reference to a weed in an area of interest, and in some instances these terms are used with reference to a crop plant in an area of interest. When used with reference to a weed, these terms may apply to only a particular type of weed or species of weed that is present or believed to be present in the area of interest.

Further provided are methods for detecting the genome edited Sorghum varieties described herein comprising obtaining a nucleic acid sample from said plant tissues, and (i) contacting said nucleic acid sample with a polynucleotide that comprises a sequence of at least 8 nucleotides that are identical to a contiguous sequence of SEQ ID NO: 1, or complements thereof; subjecting said sample and said polynucleotide to stringent hybridization conditions; and assaying said sample for hybridization of said polynucleotide to said DNA, or (ii) contacting said nucleic acid sample with a first and second PCR primer, wherein said first and second PCR primer each specifically bind to SEQ ID NO: 1, subjecting the sample to polymerase chain reaction, and assaying for an amplicon generated between said first and second primers. In certain embodiments, the first and second primer comprise SEQ ID NO: 15, 16, 17, 18, or any combination thereof.

As used herein, “stringent conditions” encompass conditions under which hybridization will only occur if there is less than 20% mismatch between the hybridization molecule and a sequence within the target nucleic acid molecule. “Stringent conditions” include further particular levels of stringency. Thus, as used herein, “moderate stringency” conditions are those under which molecules with more than 20% sequence mismatch will not hybridize; conditions of “high stringency” are those under which sequences with more than 10% mismatch will not hybridize; and conditions of “very high stringency” are those under which sequences with more than 5% mismatch will not hybridize. The following are representative, non-limiting hybridization conditions.

High Stringency condition (detects sequences that share at least 90% sequence identity): Hybridization in 5×SSC buffer (wherein the SSC buffer contains a detergent such as SDS, and additional reagents like salmon sperm DNA, EDTA, etc.) at 65° C. for 16 hours; wash twice in 2×SSC buffer (wherein the SSC buffer contains a detergent such as SDS, and additional reagents like salmon sperm DNA, EDTA, etc.) at room temperature for 15 minutes each; and wash twice in 0.5×SSC buffer (wherein the SSC buffer contains a detergent such as SDS, and additional reagents like salmon sperm DNA, EDTA, etc.) at 65° C. for 20 minutes each.

Moderate Stringency condition (detects sequences that share at least 80% sequence identity): Hybridization in 5×-6×SSC buffer (wherein the SSC buffer contains a detergent such as SDS, and additional reagents like salmon sperm DNA, EDTA, etc.) at 65-70° C. for 16-20 hours; wash twice in 2×SSC buffer (wherein the SSC buffer contains a detergent such as SDS, and additional reagents like salmon sperm DNA, EDTA, etc.) at room temperature for 5-20 minutes each; and wash twice in 1×SSC buffer (wherein the SSC buffer contains a detergent such as SDS, and additional reagents like salmon sperm DNA, EDTA, etc.) at 55-70° C. for 30 minutes each.

Non-stringent control condition (sequences that share at least 50% sequence identity will hybridize): Hybridization in 6×SSC buffer (wherein the SSC buffer contains a detergent such as SDS, and additional reagents like salmon sperm DNA, EDTA, etc.) at room temperature to 55° C. for 16-20 hours; wash at least twice in 2×-3×SSC buffer (wherein the SSC buffer contains a detergent such as SDS, and additional reagents like salmon sperm DNA, EDTA, etc.) at room temperature to 55° C. for 20-30 minutes each.

Plants produced by the methods described herein are also provided. Also provided are seeds produced or generated from any of the plants described herein, the seeds comprising an introduced genetic modification described herein, as well as seeds to produce any of the plants described herein comprising an introduced genetic modification described herein. Additionally, any genome edited Sorghum variety disclosed herein can be used to make a food or a feed product. Such methods comprise obtaining a plant, explant, seed, plant cell, or cell comprising the polynucleotide sequence and processing the plant, explant, seed, plant cell, or cell to produce a food or feed product.

The following are examples of specific embodiments of some aspects of the invention. The examples are offered for illustrative purposes only and are not intended to limit the scope of the invention in any way.

### Example 1

This example demonstrates the introduction of a targeted genetic modification to produce ACCase inhibitor resistant Sorghum.

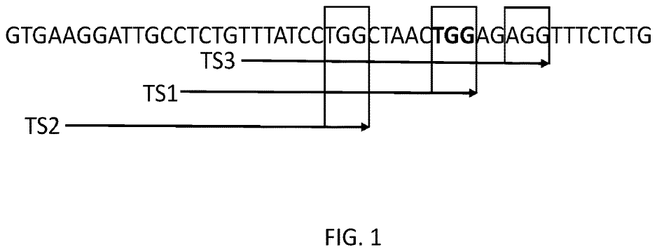

The endogenous ACCase gene was edited to produce an endogenous ACCase allele having an amino acid substitution of tryptophan to cystine at position 2032 using the gRNA/Cas9 site directed nuclease system. To modify the endogenous ACCase gene the following target sites were used: ACCase-TS1, ACCase-TS2, and ACCase-TS3 (SEQ ID NOs: 5, 6, and 7, respectively). The location of each target site in the ACCase genomic sequence (SEQ ID NO: 1) is shown in FIG. 1, and the target sequences are listed in Table 2.

TABLE 2
ACCase target site sequence

| Sorghum Genomic | | |
|---|---|---|
| Target Site | Sequence | PAM Sequence |
| ACC-TS1 | SEQ ID NO: 5 | TGG |
| ACC-TS2 | SEQ ID NO: 6 | TGG |
| ACC-TS3 | SEQ ID NO: 7 | AGG |
| ACC-TS3.1 | SEQ ID NO: 12 | AGG |

To generate the Cas9 vector the maize codon optimized Cas9 gene from Streptococcus pyogenes M1 GAS (SF370) and the potato ST-LS1 intron were used to eliminate expression in E. coli and Agrobacterium. To facilitate nuclear localization of the encoded Cas9 protein in maize cells, the Simian virus 40 (SV40) monopartite amino terminal nuclear localization signal and bipartite nuclear localization signal from VirD2 Agrobacterium tumefaciens endonuclease were incorporated at the amino and carboxyl-terminal of the Cas9 open reading frame, respectively. The Cas9 gene was operably linked to a maize Ubiquitin promoter (Zm-Ubi pro) and potato intron II (PinII) using standard molecular biological techniques.

To direct the Cas9 nuclease to the designated genomic target sites, a maize U6 polymerase III promoter (SEQ ID NO: 19) and its cognate U6 polymerase III termination sequence was used to direct initiation and termination of gRNA expression. The guide RNA (SEQ ID NO: 13) was an engineered crRNA-tracrRNA fusion transcript, comprising the 20 bp variable targeting domain which was chosen from a maize genomic target site. The guide RNA variable targeting domains for ACCase gene editing were identified as ACC-CR1 (SEQ ID NO: 8), ACC-CR2 (SEQ ID NO: 9), and ACC-CR3 (SEQ ID NO: 10). The guide RNA variations ACC-CR3.1 (SEQ ID NO: 11) and ACC-TS3.1 (SEQ ID NO:12), were also included to increase cleavage and editing frequency. DNA encoding each of the variable nucleotide targeting domains was cloned into a gRNA expression cassette through BsbI sites.

The donor of the substitution/repair template for ACC-TS3 contained the nucleotide substitution to produce an ACCase gene encoding a polypeptide comprising the W2032C amino acid substitution along with a 100 bp homology arm 1 and a 103 bp homology arm 2 sequence flanking ACC-TS3 (SEQ ID NO: 7), ACC-TS3.1 was placed outside of the homology arms to increase editing efficiency in Agrobacterium stable transformation.

Agrobacterium auxotrophic strain LBA4404 Thy-carrying a ternary vector was selected as the delivery system to generate the ACCase editing events. The ternary vector system contains the T-DNA binary vector and pVIR assessor plasmid. The T-DNA binary vector had multi-gene cassettes, containing the Zm-Ubi pro: SpCas9, Zm-U6 pro: guide RNAs, donor repair template with desired edit, additional gene cassettes including Zm-Ubi pro: NPTII (neomycin phosphotransferase II), maize morphogenic genes Zm-Pltp pro: Bbm (Baby boom) and Zm-Axig1 pro: WUS2 (Wuschel2) for selection of transformants and to stimulate transformation. The ternary design was assembled by first mobilizing the accessory plasmid in the Agrobacterium auxotrophic strain LBA4404 Thy- and selecting on media supplemented with gentamycin (25 mgl-1). Subsequently, the binary constructs were electroporated into Agrobacterium strain LBA4404 Thy-containing the accessory plasmid and recombinant colonies were selected on media supplemented with both gentamycin and spectinomycin. All constructs were then subjected to next-generation sequencing and sequence confirmation before conducting transformation experiments.

To select one guide for stable transformation, the three guides described above were transiently tested using ribonucleoprotein (RNP) and plasmid DNA particle bombardment. Briefly, S. pyogenes Cas9 protein with two nuclear localization signals was received from the internal Corteva sources, sgRNA in the form of RNA molecules was synthesized in Synthego. To generate a guide RNA-Cas9 ribonucleoprotein (RNP) complex, 7 μg of Cas9 protein and 3 μg of gRNA molecules (1:2 molar ratio) were mixed in 1×NEB Buffer 3 and 1 μl of RNA inhibitor (Ribo Guard, Epicentre, USA) to a total volume of 20 μl and incubated at room temperature for 15 min. The particle delivery matrix comprised of the RNP complexes complemented with plasmids containing Ubiquitin promoter-regulated YFP (20 ng), PLTP-regulated ODP2 (10 ng), and maize auxig promoter-regulated WUS (10 ng) were delivered into Sorghum embryo cells. Briefly, the RNPs and DNA were precipitated onto 0.6 μm (average diameter) gold particles (Bio-Rad, USA) using a water soluble cationic lipid TransIT-2020 (Mirus, USA) as follows: 50 μl of gold particles (water suspension of 10 mg ml-1) and 2 μl of TransIT-2020 water solution were added to the premixed RNPs and DNA vectors, mixed gently, and incubated on ice for 10 min. RNP/DNA-coated gold particles were then pelleted in a microfuge at 8,000 g for 30 s and supernatant was removed. The pellet was resuspended in 50 μl of sterile water by brief sonication. Immediately after sonication, coated gold particles were loaded onto a macrocarrier (10 μl each) and allowed to air dry. Immature Sorghum embryos, 8-10 days after pollination, were bombarded using a PDS-1000/He Gun (Bio-Rad, USA) with a rupture pressure of 425 pounds per inch square. Embryos were harvested at 3 days after bombardment.

Immature embryo explants isolated from the Sorghum plants were transformed with Agrobacterium auxotrophic strain LBA4404 Thy-carrying a ternary vector transformation system to generate transgenic Sorghum plants. Nine-weeks callus proliferation phase was eliminated due to morphogenic gene cassettes used in the vector. Following Agrobacterium infection and co-cultivation of the immature embryos, the immature embryos were then sub-cultured on multi-purpose medium with selection (250 mg/1-G418) for three weeks to induce somatic embryo formation. After the three-week-long G418 selection, somatic embryos were transferred to the maturation medium without selection for four weeks before rooting to produce T0 plants.

DNA was extracted from the embryos after bombardment, which then went through PCR and amplicon deep sequencing with the primers shown in Table 3. High target site mutation frequency was obtained with ACC-CR3, and the most dominant type of mutation at the ACC-CR3 target site was referred to as ACC-TS3.1, the corresponding guide RNA of ACC-CR3.1 to the new target site was included in the agro vector.

TABLE 3
Primers used in guide selection and edit
event screening

| Primer name | SEQ ID NO: |
|---|---|
| ACC-TS3F | 15 |
| ACC-TS3R | 16 |
| ACC-editF | 17 |
| ACC-editR | 18 |

To identify ACCase edited variants, genomic DNA was extracted from leaf tissue of the T0 plants. Next generation Sequencing (NGS) (Illumina) was used to identify the edited TO variants. The region was PCR amplified and the primers used in the primary PCR reaction are shown in Table 3. One T0 plant with the W2032C edit was obtained from the 271 analyzed TO plants.

The ACCase edited T0 plant was transferred to a flat in a controlled environment. The TO plant was selfed to produce T1 (S1) seeds, only 24 T1 seeds were produced from the edited TO plant. T1 (S1) plants went through comprehensive molecular characterization to not only confirm that edits observed in T0 plant were stably inherited but also to verify that the T1 plants were free of any part of the T-DNA used during transformation process. The same NGS assay for identifying edited variants used at the TO stage was performed on T1 plant samples to confirm the presence of the edit. qPCR was performed on all helper genes in the T-DNA, which included Cas9, guide RNA, transformation selection marker (NPTII), and the transformation enhancing genes Bbm and WUS2 to confirm that the genes (T-DNA) segregated away from the generated edited alleles. Selected T1 plants with edit and null segregants were analyzed further with Southern by Sequencing (SbS) to further support that the plants were free of any foreign DNA. All survived T1 seeds/seedlings were homozygous with Trp2032Cys edit and free of T-DNA.

### Example 2

This example demonstrates the tolerance of the ACCase edited plants ACCase inhibitors.

ACCase edited T1 seeds were planted in flats, all seeds germinated and were heathy looking. Additionally, they were all able to produce T2 seeds with high seed count setting. Nineteen homozygous ACCase edited T1 plants at the 8-leaf stage were sprayed with quizalofop-p-ethyl at a rate of 7 fl. ounce (0.034-0.083 lb ai), which is the average dose from the Assure II label. All 19 homozygous ACCase edited T1 plants showed resistance (tolerance) to the herbicide. A subset of the T1 plants after the spray test were kept to maturity and all produced T2 seeds with good seed setting. In contrast, the wild-type control plants were all susceptible to the quizalofop-p-ethyl spray and died.

This example demonstrates that the ACCase edited Sorghum plants had tolerance to an ACCase inhibitor.

### Example 3

This example demonstrates field tolerance of the ACCase edited plants.

The ACCase edited plants generated in Example 1 were outcrossed with 3 male sterile lines in a controlled environment. The F1 plants which contain a single copy of the W2032C edit in the ACCase gene and the edited elite inbred line containing 2 copies of the W2032C ACCase (hereinafter referred to as inbred_W3032C) along with the non-edited elite inbred (wt control) and the 3 male sterile lines were tested in the field for efficacy. Four short rows, each row with ~100 seedlings, were planted for each variety in replicate. Once the plants reached the 5-6 leaves stage, replicate 1 for each variety was treated with a 1× dose of Assure II (Quizalofop-P-ethyl) and replicate 2 for each variety was treated with a 2× dose of Assure II (Quizalofop-P-ethyl). Plants were then scored for tolerance at 7- and 14-days post application. The plots were scored by giving a 1-9 score to each plot, with 1 being dead and 9 no visible response. As shown in Table 4, the inbred_W2032C plants showed tolerance to the herbicide Quizalofop-P-ethyl, control plants without the W2032C ACCase edit showed no tolerance with all plants dead at 10-14 days post spray. The three F1 hybrids with a single copy of the W2032C ACCase edit showed some tolerance to the herbicide Quizalofop-P-ethyl.

The PH2545MW_W2032C was given score of 7 with 1× dose of Quizalofop-P-ethyl application (Table 4), as some twisting of the leaves due to herbicide injury was observed, however, after a week or so the twisting of the leaves may not be visible anymore because they were growing out of it, which may be indicated by the score change from 5 to 6 at day 7 and day 14 post 2× dose of Quizalofop-P-ethyl application.

TABLE 4
Field Test Scores for Sorghum Varieties Treated with 1x or 2x Quizalofop-P-ethyl

| | | Quizalofop- | 7-day Post | 14-day Post |
|---|---|---|---|---|
| | ACCaseW2032C | P-ethyl | Application | Application |
| Variety name | copy number | Dosage | Score | Score |
| elite inbred_WT | 0 | 1x | 3 | 1 |
| elite inbred_W2032C | 2 | 1x | 7 | 7 |
| Male Sterile 1_WT | 0 | 1x | 2 | 1 |
| F1 Hybrid 1_W2032C | 1 | 1x | 5 | 4 |
| Male Sterile 2_WT | 0 | 1x | 2 | 1 |
| F1 Hybrid 2_W2032C | 1 | 1x | 5 | 4 |
| Male Sterile 3_WT | 0 | 1x | 3 | 1 |
| F1 Hybrid 3_W2032C | 1 | 1x | 5 | 3 |
| elite inbred_WT | 0 | 2x | 3 | 1 |
| elite inbred _W2032C | 2 | 2x | 5 | 6 |
| Male Sterile 1_WT | 0 | 2x | 2 | 1 |
| F1 Hybrid 1_W2032C | 1 | 2x | 4 | 3 |
| Male Sterile 2_WT | 0 | 2x | 2 | 1 |
| F1 Hybrid 2_W2032C | 1 | 2x | 5 | 3 |
| Male Sterile 3_WT | 0 | 2x | 2 | 1 |
| F1 Hybrid 3_W2032C | 1 | 2x | 5 | 4 |

All publications and patent applications in this specification are indicative of the level of ordinary skill in the art to which this invention pertains. All publications and patent applications are herein incorporated by reference to the same extent as if each individual publication or patent application was specifically and individually indicated by reference.

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

1. A genome edited Sorghum variety comprising an introduced genetic modification at an endogenous acetyl-CoA carboxylase (ACCase) gene to encode a modified ACCase polypeptide that is at least 95% identical to SEQ ID NO: 2 and comprises a cysteine at a position corresponding to position 2032 of SEQ ID NO: 2, the edited Sorghum variety having increased tolerance to at least one ACCase inhibitor herbicide as compared to a control Sorghum variety not comprising the introduced genetic modification.

2. (canceled)

3. The genome edited Sorghum variety of claim 1, wherein the Sorghum variety is an elite inbred Sorghum variety or an elite Sorghum hybrid variety.

4-6. (canceled)

7. The genome edited Sorghum variety of any one of claim 3, wherein the genome edited Sorghum variety is an elite hybrid Sorghum variety and has a yield of at least 95 bushels per acre when grown in the presence of the at least one ACCase inhibitor herbicide.

8-10. (canceled)

11. The genome edited Sorghum variety of claim 1, wherein the edited Sorghum variety has tolerance to quizalofop-p-ethyl herbicide when applied at a rate of at least 0.05 pounds of active ingredient per acre (lb ai/A).

12. A seed produced by the genome edited Sorghum variety of claim 1, the seed comprising the introduced genetic modification.

13. A genome edited Sorghum hybrid comprising an introduced genetic modification at an endogenous acetyl-CoA carboxylase (ACCase) gene to encode a modified ACCase polypeptide that is at least 95% identical to SEQ ID NO: 2 and comprises a cysteine at a position corresponding to position 2032 of SEQ ID NO: 2, the edited Sorghum hybrid having increased tolerance to at least one ACCase inhibitor herbicide as compared to a control Sorghum hybrid not comprising the introduced genetic modification.

14-16. (canceled)

17. The genome edited Sorghum hybrid of claim 13, wherein the yield of the edited Sorghum hybrid is at least 95 bushels per acre when grown in the presence of the at least one ACCase inhibitor herbicide.

18. The genome edited Sorghum hybrid of claim 13, wherein the introduced genetic modification is present on a single allele of the ACCase gene.

19. (canceled)

20. The genome edited Sorghum hybrid of claim 13, wherein the edited Sorghum hybrid has tolerance to quizalofop-p-ethyl herbicide when applied at a rate of at least 0.05 pounds of active ingredient per acre (lb ai/A).

21. A seed produced by the genome edited Sorghum hybrid of claim 13, the seed comprising the introduced genetic modification.

22. A method for producing the ACCase inhibitor tolerant Sorghum hybrid of claim 13, the method comprising crossing a first Sorghum variety comprising an introduced genetic modification at an endogenous acetyl-CoA carboxylase (ACCase) gene to encode a modified ACCase polypeptide that is at least 95% identical to SEQ ID NO: 2 and comprises a cysteine at a position corresponding to position 2032 of SEQ ID NO: 2, with a second Sorghum variety.

23-25. (canceled)

26. The method of claim 22, wherein the second Sorghum variety comprises an introduced genetic modification at an endogenous acetyl-CoA carboxylase (ACCase) gene to encode a modified ACCase polypeptide that is at least 95% identical to SEQ ID NO: 2 and comprises a cysteine at a position corresponding to position 2032 of SEQ ID NO: 2.

27-34. (canceled)

35. A method for producing an ACCase inhibitor tolerant Sorghum variety the method comprising:
a. providing to a plant cell comprising an ACCase gene sequence a guide RNA, a polynucleotide modification template comprising at least one nucleotide modification of the ACCase gene sequence to encode a modified ACCase polypeptide that is at least 95% identical to SEQ ID NO: 2 and comprises a cysteine at a position corresponding to position 2032 of SEQ ID NO: 2, and a Cas endonuclease, the guide RNA and Cas endonuclease capable of forming a complex that enables the Cas endonuclease to introduce a double strand break at a target site sequence in the ACCase gene of said plant cell;
b. obtaining a plant from the plant cell of (a);
c. evaluating the plant of (b) for the presence of said at least one nucleotide modification; and
d. selecting a progeny Sorghum plant comprising the modified ACCase gene and having increased tolerance to ACCase inhibitor herbicides as compared to a control plant not comprising the modification.

36-37. (canceled)

38. The method of claim 35, wherein the target site sequence in the ACCase gene comprises a nucleotide sequence that is at least 95% identical to any one of SEQ ID NOs: 5, 6, 7, or 12.

39. The method of claim 35, wherein the guide RNA comprises a crRNA-tracrRNA fusion transcript, the tracrRNA comprising a nucleotide sequence that is at least 95% identical to SEQ ID NO: 13 and the crRNA comprising a nucleotide sequence that is at least 95% identical to any one of SEQ ID NOs: 8, 9, or 10.

40-41. (canceled)

42. The method of claim 35, wherein the polynucleotide modification template comprises a nucleotide sequence that is at least 95% identical to SEQ ID NO: 14.

43. The method of claim 35, wherein further comprises crossing the selected progeny Sorghum plant with a second Sorghum plant to produce an F1 progeny plant.

44. (canceled)

45. A plant produced by the method of claim 35, wherein the plant comprises the modified ACCase gene and has increased tolerance to ACCase inhibitor herbicides as compared to a control plant not comprising the modification and a yield that is greater than or equal to the yield of the control Sorghum hybrid when grown under the same environmental conditions.

46-53. (canceled)

54. The genome edited Sorghum variety of claim 1, wherein the genome edited Sorghum variety further comprises at least one additional polynucleotide that confers tolerance to glyphosate, 2,4-D, glufosinate, or any combination thereof.

55-57. (canceled)

58. The genome edited Sorghum hybrid of claim 13, wherein the genome edited Sorghum variety further comprises at least one additional polynucleotide that confers tolerance to glyphosate, 2,4-D, glufosinate, or any combination thereof.

59-60. (canceled)
